(19) United States
(12) Patent Application Publication
(10) Pub. No.: US 2018/0161438 A1
(43) Pub. Date: Jun. 14, 2018

(54) Aqueous Ophthalmic Composition

(71) Applicant: ROHTO PHARMACEUTICAL CO., LTD., Osaka (JP)

(72) Inventor: Yasuko Matsumura, Osaka (JP)

(73) Assignee: ROHTO PHARMACEUTICAL CO., LTD., Osaka (JP)

(21) Appl. No.: 15/577,560

(22) PCT Filed: May 24, 2016
(86) PCT No.: PCT/JP2016/065317

§ 371 Date: Nov. 28, 2017

(30) Foreign Application Priority Data
May 28, 2015 (JP) 2015-108837
Sep. 30, 2015 (JP) 2015-193310

(51) Int. Cl. A61K 47/44; A61K 9/08; A61K 9/00; A61K 9/06; A61K 47/32; A61K 47/38; A61K 47/02; A61K 47/10; A61K 47/36; A61K 47/18; A61K 47/22; A61K 31/27; A61K 31/4164; A61K 31/4174; A61K 31/635; A61K 47/34; A61K 47/26; A61K 47/20; A61K 47/12

(52) U.S. Cl. CPC A61K 47/44; A61K 47/12; A61K 9/0048; A61K 9/06; A61K 47/32; A61K 47/38; A61K 47/02; A61K 47/10; A61K 47/36; A61K 47/186; A61K 47/18; A61K 47/22; A61K 31/27; A61K 31/4164; A61K 31/4174; A61K 31/635; A61K 47/34; A61K 47/26; A61K 47/20; A61K 9/08

(57) ABSTRACT

The present invention relates to an aqueous ophthalmic composition that contains (A) vaseline and (B) at least one selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil.

### TECHNICAL FIELD

The present invention relates to an aqueous ophthalmic composition.

### BACKGROUND ART

Possible causes of discomfort in the eyes include dry eye, invasion of foreign substances, and foreign body sensation due to impurities on contact lenses, attachment of pollen, medicinal properties or the like in individuals wearing contact lenses. In order to reduce those factors, various technologies are disclosed.

For example, for dry eye, there is disclosed a pharmaceutical composition containing human serum albumin produced by a recombinant yeast obtained by transforming yeast with a gene encoding human serum albumin as an active component (Patent Literature 1). Further, regarding the case of wearing contact lenses, in ionic silicone hydrogel lenses, there is disclosed an ophthalmic composition for silicone hydrogel lenses in which vitamin B12 is used in combination with at least one selected from the group consisting of chlorpheniramine, glicyrrhizic acid, and salts thereof and which can reduce the accumulation of proteins of the pollen attached to the contact lenses (Patent Literature 2).

However, there is a strong need for a method of effectively reducing eye discomfort.

### CITATION LIST

### Patent Literature

Patent Literature 1: Japanese Unexamined Patent Publication No. 2014-205705

Patent Literature 2: Japanese Unexamined Patent Publication No. 2014-205717

### SUMMARY OF INVENTION

### Problems to be Solved by the Invention

An object of the present invention is to provide an aqueous ophthalmic composition capable of reducing ocular discomfort and uncomfortable feeling.

### Means for Solving the Problems

In order to solve the above problems, the present inventors have been dedicated to making repetitive studies and found out that fluidity of the liquid, such as tear fluid, on the eye surface is deeply involved in ocular discomfort, and an improvement of this fluidity leads to a reduction in discomfort on the eye surface.

Further, the present inventors have found a new problem that when vaseline is singly used as the component of the aqueous ophthalmic composition, the dynamic contact angle (also referred to as “angle of advance”) is increased and the fluidity of the liquid is reduced, thereby increasing ocular discomfort, particularly, discomfort during use of contact lenses.

Further, the present inventors have unexpectedly found that a combination of (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil as the components of the aqueous ophthalmic composition results in an improvement in the dynamic contact angle whereby the fluidity of the liquid is increased and ocular discomfort, when blinking for example, is effectively reduced. The present invention is based on this knowledge.

The present invention relates to, for example, the following inventions.

[1] An aqueous ophthalmic composition comprising: (A) vaseline; and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil.

[2] The aqueous ophthalmic composition according to [1], wherein (A) the vaseline is white vaseline.

[3] The aqueous ophthalmic composition according to [1] or [2], further comprising (C) a nonionic surfactant.

[4] The aqueous ophthalmic composition according to any one of [1] to [3], further comprising (D) a buffer.

[5] The aqueous ophthalmic composition according to any one of [1] to [4], wherein the content of (A) the vaseline is from 0.00001 to 10 w/v % based on the total amount of the aqueous ophthalmic composition.

[6] The aqueous ophthalmic composition according to any one of [1] to [5], wherein the composition is used for contact lenses.

[7] The aqueous ophthalmic composition according to any one of [1] to [6], wherein the composition is used for reducing ocular discomfort.

[8] The aqueous ophthalmic composition according to [7], wherein the composition is used for reducing discomfort during wearing contact lenses.

[9] The aqueous ophthalmic composition according to any one of [1] to [8], wherein the saccharide is methyl cellulose, hydroxyethyl cellulose, hydroxypropylcellulose, hydroxypropylmethylcellulose, carboxymethyl cellulose, hyaluronic acid, chondroitin sulfate, alginic acid, dextran, gellant gum, salts thereof and glucose;

the vinyl-based polymer compound is polyvinyl alcohol, polyvinylpyrrolidone, and a carboxyvinyl polymer;

the polyoxyethylene polyoxypropylene glycol is Poloxamer 188 and Poloxamer 407;

the polyalcohol is glycerin, propylene glycol, polyethylene glycol, and mannitol;

the inorganic salt is sodium chloride, potassium chloride, calcium chloride, zinc chloride, and magnesium sulfate;

the vegetable oil is sesame oil and castor oil;

the amino acid is aspartic acid, aminoethylsulfonic acid, and salts thereof;

the vitamin is flavin adenin dinucleotide sodium, panthenol, sodium pantothenate, calcium pantothenate, pyridoxine hydrochloride, cyanocobalamin, retinol acetate, retinol palmitate, and tocopherol acetate;

the ocular muscle modulator component is neostigmine and a salt thereof;

the vasoconstrictor is tetryzoline, naphazoline, and salts thereof;

the preservative is alkyldiaminoethylglycine hydrochloride, benzalkonium chloride, benzethonium chloride, chlorhexidine glyconate, chlorobutanol, sorbic acid, potassium sorbate, sodium dehydroacetate, methyl parahydroxybenzoate, ethyl parahydroxybenzoate, propyl parahydroxybenzoate, butyl parahydroxybenzoate, and a biguanide compound; and

the stabilizer is dibutylhydroxytoluene, trometamol, and monoethanolamine; and

the sulfa drug is sulfisomezole and sulfamethoxazole sodium.

[10] The aqueous ophthalmic composition according to any one of [1] to [9], wherein the total content of the component (B) is from 0.00001 to 50000 parts by weight based on 1 part by weight of the total content of the component (A).

[11] The aqueous ophthalmic composition according to any one of [1] to [10], comprising 50% by weight or more of water relative to the total amount of the aqueous ophthalmic composition.

[12] The aqueous ophthalmic composition according to any one of [1] to [11], wherein the composition is contained in a plastic container.

### Effects of the Invention

Since the aqueous ophthalmic composition of the present invention is obtained by combining (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil, the dynamic contact angle is improved and the fluidity of the liquid on the ocular surface is increased, thereby producing an effect of more effectively reducing ocular discomfort, for example when blinking (particularly, discomfort during use of contact lenses).

### EMBODIMENTS FOR CARRYING OUT THE INVENTION

Hereinafter, embodiments for executing the present invention will be explained in detail. However, the present invention is not limited to the following embodiments.

The term “aqueous ophthalmic composition” used herein means an ophthalmic composition containing water. The content of water in the aqueous ophthalmic composition is preferably 20% by weight or more, more preferably 30% by weight or more, still more preferably 50% by weight or more, still more preferably 70% by weight or more, still more preferably 80% by weight or more, further still more preferably 85% by weight or more, further still more preferably 90% by weight or more, further still more preferably 95% by weight or more, and particularly preferably 97.5% by weight or more relative to the total amount of the aqueous ophthalmic composition. Note that “% by weight” is synonymous with “w/v %”.

Unless indicated otherwise herein, the unit “%” of content means “w/v %” and is synonymous with “g/100 mL”.

Unless indicated otherwise herein, the abbreviation “POE” means polyoxyethylene.

Unless indicated otherwise herein, the abbreviation “POP” means polyoxypropylene.

[1. Aqueous Ophthalmic Composition]

The aqueous ophthalmic composition according to the present embodiment contains (A) vaseline (simply referred to as “component (A)”) and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil (simply referred to as “component (B)”).

The term “vaseline” used herein includes “yellow vaseline” obtained by purifying a mixture of hydrocarbon compounds from petroleum and “white vaseline” purified by decolorization.

Commercially available vaseline can be used without any particular limitation. Specific examples of the vaseline include Perfecta, Protopet Alba, Protopet White 1S, White Fonoline, Protopet White 2L, Protopet White 3C, Yellow Fonoline, Protopet Yellow 1E, Protopet Yellow 2A, and Protopet Super White (all manufactured by Witco), Penreco Ultima, Penreco Super, Penreco Snow, Penreco Regent, Penreco Lily, Penreco Cream, Penreco Royal, Penreco Blond, Penreco Amber, Penreco4650, Penreco Snow V, and Ointment BaseNo. 4, No. 6, No. 8 (all manufactured by Penreco), Perlatum330, Perlatum310/410, Perlatum320/420, Perlatum321, Perlatum325/425, and Perlatum325/415 (all manufactured by IGI), Snowwhite series such as Snowwhite Spetial and S nowwhite A4, Microwax MA, Sonnecone CM, Sonnecone DM, White Fonoline H, and White Protopet1 SH (all manufactured by Sonneborn), white vaseline listed in Japanese Pharmacopoeia (manufactured by Maruishi Pharmaceutical Co., Ltd., manufactured by NIKKO Pharmaceutical Co., Ltd), yellow vaseline listed in Japanese Pharmacopoeia (manufactured by Maruishi Pharmaceutical Co., Ltd., manufactured by NIKKO Pharmaceutical Co., Ltd), Crolatum V (manufactured by Croda Japan KK), Sunwhite P-1, Sunwhite P-150, Sunwhite P-200, and Sunwhite S-200 (all manufactured by NIKKO RICA CORPORATION.), Nomucoat W (manufactured by Nisshin Oillio Group), Propeto (manufactured by Maruishi Pharmaceutical Co., Ltd.), and white vaseline (manufactured by Crompton). Further, those obtained by purifying the above vaseline may be used. Such vaseline may be used singly, or in an arbitrary combination of two or more kinds thereof.

Among them, white vaseline prescribed by the Japanese Pharmacopoeia, 16th revision is preferred from the viewpoint of more significantly exerting the effect according to the present invention.

The content of the component (A) in the aqueous ophthalmic composition according to the present embodiment is not particularly limited and is appropriately set depending on the use and dosage form of the aqueous ophthalmic composition. Regarding the lower limit of the content of the component (A), from the viewpoint of more significantly exerting the effect according to the present invention, the total content of the component (A) may be 0.00001 w/v % or more, preferably 0.0001 w/v % or more, more preferably 0.0005 w/v % or more, and still more preferably 0.001 w/v % or more, for example, based on the total amount of the aqueous ophthalmic composition. Although the upper limit of the content of the component (A) is not particularly limited, the total content of the component (A) may be 5 w/v % or less, preferably 1 w/v % or less, more preferably 0.5 w/v % or less, still more preferably 0.25 w/v % or more, yet still more preferably 0.1 w/v % or less, and particularly preferably 0.05 w/v % or less, for example, based on the total amount of the aqueous ophthalmic composition. These upper and lower limits can be arbitrarily combined.

Regarding a specific content range of the component (A), from the viewpoint of more significantly exerting the effect according to the present invention, the total content of the component (A) may be from 0.00001 to 10 w/v %, preferably from 0.0001 to 5 w/v %, more preferably from 0.0001 to 1 w/v %, still more preferably from 0.0005 to 0.5 w/v %, yet still more preferably from 0.0005 to 0.25 w/v %, yet still preferably from 0.001 to 0.1 w/v %, and particularly preferably from 0.001 to 0.05 w/v %, for example, based on the total amount of the aqueous ophthalmic composition.

The component (B) is at least one selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil. The component (B) may be used singly, or in combination of two or more kinds thereof.

Among the component (B), the vinyl-based polymer compound, saccharide, amino acid, inorganic salt compound, and preservative are preferred, the vinyl-based polymer compound and saccharide are more preferred, and the saccharide is still more preferred, from the viewpoint of more significantly exerting the effect according to the present invention.

Regarding the content of the component (B) in the aqueous ophthalmic composition according to the embodiment, from the viewpoint of further improving the effect according to the present invention, the total content of the component (B) is usually from 0.0000001 to 25 w/v %, preferably from 0.0000001 to 10 w/v %, more preferably from 0.000001 to 8 w/v %, still more preferably from 0.000005 to 5 w/v %, yet still more preferably 0.00001 to 3 w/v %, particularly preferably from 0.0001 to 2 w/v %, and most preferably from 0.0001 to 1 w/v %, for example, based on the total amount of the aqueous ophthalmic composition.

The content ratio of the component (B) to the component (A) in the aqueous ophthalmic composition according to the present embodiment is not particularly limited, and it is appropriately set depending on the kind of the component (A), the component (B) or the like. Regarding the content ratio of the component (B) to the component (A), from the viewpoint of further improving the effect according to the present invention, the total content of the component (B) is usually from 0.00001 to 50000 parts by weight, preferably from 0.0001 to 50000 parts by weight, more preferably from 0.0001 to 30000 parts by weight, still more preferably from 0.0001 to 20000 parts by weight, yet still more preferably from 0.0001 to 10000 parts by weight, particularly preferably from 0.0001 to 5000 parts by weight, and most preferably from 0.001 to 5000 parts by weight, for example, based on 1 part by weight of the total content of the component (A) contained in the aqueous ophthalmic composition according to the present embodiment.

Examples of vinyl-based polymer compounds include vinyl alcohol-based polymers such as polyvinyl alcohol (completely or partially saponificated polyvinyl alcohol), vinylpyrrolidone-based polymers such as polyvinylpyrrolidone and carboxyvinyl polymers. Commercially available vinyl-based polymer compounds can also be used.

Among the vinyl-based polymer compounds, polyvinylpyrrolidone K17, polyvinylpyrrolidone K25, polyvinylpyrrolidone K30, polyvinylpyrrolidone K90, polyvinyl alcohol (a partially saponificated polyvinyl alcohol) or a carboxyvinyl polymer is preferred, polyvinylpyrrolidone K25, polyvinylpyrrolidone K90 or a carboxyvinyl polymer is more preferred, and polyvinylpyrrolidone K25 and polyvinylpyrrolidone K90 are still more preferred, from the viewpoint of further improving the effect according to the present invention.

The vinyl-based polymer compounds may be used singly, or in combination of two or more kinds thereof.

The content of the vinyl-based polymer compound in the aqueous ophthalmic composition according to the present embodiment is appropriately set depending on the kind and molecular weight of the compound. Regarding the content of the vinyl-based polymer compound, from the viewpoint of further improving the effect according to the present invention, the total content of the vinyl-based polymer compound is usually from 0.001 to 25 w/v %, preferably from 0.001 to 10 w/v %, more preferably from 0.005 to 5 w/v %, and still more preferably from 0.01 to 3 w/v %, for example, based on the total amount of the aqueous ophthalmic composition.

In the case of using polyvinylpyrrolidone as the component (B), in view of more significantly exerting the effect according to the present invention, the content of polyvinylpyrrolidone is usually from 0.001 to 25 w/v %, preferably from 0.001 to 10 w/v %, and more preferably from 0.01 to 3 w/v %, for example, based on the total amount of the aqueous ophthalmic composition.

In the case of using a carboxyvinyl polymer as the component (B), in view of more significantly exerting the effect according to the present invention, the content of the carboxyvinyl polymer is usually from 0.001 to 3 w/v %, preferably from 0.001 to 1 w/v %, and more preferably from 0.01 to 0.5 w/v %, for example, based on the total amount of the aqueous ophthalmic composition.

Examples of saccharides include polysaccharides (e.g., a cellulose-based polymer compound, an acid mucopolysaccharide, dextran, gellant gum, and salts thereof), oligosaccharides (e.g., cyclodextrin), and monosaccharides (e.g., glucose). Commercially available saccharides can also be used. The saccharides may be used singly, or in combination of two or more kinds thereof.

In the case of using a saccharide as the component (B), a polysaccharide and glucose are preferred from the viewpoint of further improving the effect according to the present invention. Among them, a cellulose-based polymer compound, an acid mucopolysaccharide, dextran, gellant gum, glucose are more preferred.

The content of the saccharide in the aqueous ophthalmic composition according to the present embodiment is appropriately set depending on the kind and molecular weight of the compound. Regarding the content of the saccharide, from the viewpoint of further improving the effect according to the present invention, the total content of the saccharide is usually from 0.00001 to 25 w/v %, preferably from 0.00005 to 10 w/v %, more preferably from 0.0001 to 10 w/v %, still more preferably from 0.0005 to 7 w/v %, and yet still more preferably from 0.001 to 5 w/v %, for example, based on the total amount of the aqueous ophthalmic composition.

As the cellulose-based polymer compound, a cellulose-based polymer compound obtained by substituting a hydroxyl group of cellulose by another functional group can be used. Examples of functional groups substituting the hydroxyl group of cellulose include methoxy, ethoxy, hydroxymethoxy, hydroxyethoxy, hydroxypropoxy, carboxymethoxy, and carboxyethoxy groups. Commercially available cellulose-based polymer compounds can also be used.

Examples of cellulose-based polymer compounds include methyl cellulose, ethyl cellulose, hydroxyethyl cellulose, hydroxymethyl cellulose, hydroxypropylcellulose, hydroxypropylmethylcellulose (hydroxypropylmethylcellulose), carboxymethyl cellulose, carboxyethyl cellulose and salts thereof. Here, the salts may be medically, pharmacologically (pharmaceutically) or physiologically acceptable salts. Among them, an alkali metal salt is preferred, a sodium salt, a potassium salt, and the like are more preferred.

Among cellulose-based polymer compounds, methyl cellulose, hydroxyethyl cellulose, hydroxypropylmethylcellulose (2208, 2906, 2910), hydroxypropylcellulose, carboxymethyl cellulose or salts thereof are preferred, hydroxypropylmethylcellulose, hydroxyethyl cellulose or carboxymethylcellulose sodium is more preferred, and hydroxypropylmethylcellulose, hydroxyethyl cellulose or carboxymethylcellulose sodium is yet still more preferred from the viewpoint of further improving the effect according to the present invention. Further, in view of more significantly exerting the effect according to the present invention, hydroxypropylmethylcellulose is preferably hydroxypropylmethylcellulose 2906 or hydroxypropyl cellulose 2910.

The cellulose-based polymer compounds may be used singly, or in combination of two or more kinds thereof.

The content of the cellulose-based polymer compound in the aqueous ophthalmic composition according to the present embodiment is appropriately set depending on the kind and molecular weight of the compound or the like. Regarding the content of the cellulose-based polymer compound, from the viewpoint of further improving the effect according to the present invention, the total content of the cellulose-based polymer compound is usually from 0.0001 to 25 w/v %, preferably from 0.001 to 10 w/v %, more preferably from 0.001 to 5 w/v %, still more preferably from 0.002 to 4 w/v %, and still more preferably from 0.01 to 2 w/v %, for example, based on the total amount of the aqueous ophthalmic composition.

In the case of using hydroxypropylmethylcellulose as the component (B), in view of more significantly exerting the effect according to the present invention, the content of hydroxypropylmethylcellulose is usually from 0.0001 to 5 w/v %, preferably from 0.0005 to 4 w/v %, more preferably from 0.001 to 3 w/v %, still more preferably, 0.002 to 2 w/v %, and yet still more preferably from 0.01 to 1.5 w/v %, for example, based on the total amount of the aqueous ophthalmic composition.

In the case of using hydroxyethylcellulose as the component (B), in view of more significantly exerting the effect according to the present invention, the content of hydroxyethyl cellulose is usually from 0.0001 to 3 w/v %, preferably from 0.001 to 1 w/v %, and more preferably from 0.01 to 0.8 w/v %, for example, based on the total amount of the aqueous ophthalmic composition.

In the case of using carboxymethyl cellulose or a salt thereof as the component (B), in view of more significantly exerting the effect according to the present invention, the content of carboxymethylcellulose sodium is usually from 0.0001 to 3 w/v %, preferably from 0.001 to 1.5 w/v %, and more preferably from 0.01 to 0.5 w/v %, for example, based on the total amount of the aqueous ophthalmic composition.

Examples of acid mucopolysaccharides include hyaluronic acid, chondroitin sulfate, and alginic acid.

Among the acid mucopolysaccharides, a hyaluronic acid or a salt thereof, chondroitin sulfate or a salt thereof; an alginic acid or a salt thereof is preferred, chondroitin sulfate or a salt thereof and an alginic acid or a salt thereof are more preferred, and chondroitin sulfate or salt is still more preferred in view of more significantly exerting the effect according to the present invention. The salt of hyaluronic acid salt, the salt of chondroitin sulfate or the salt of alginic acid is not particularly limited as long as it is a medically, pharmacologically (pharmaceutically) or physiologically acceptable salt. As the salt of hyaluronic acid, the salt of chondroitin sulfate or the salt of alginic acid, for example, an alkali metal salt is preferred, and a sodium salt, a potassium salt or the like is more preferred, and a sodium salt is still more preferred.

The acid mucopolysaccharides may be used singly, or in combination of two or more kinds thereof.

The content of the acid mucopolysaccharide in the aqueous ophthalmic composition according to the present embodiment is appropriately set depending on the kind and molecular weight of the compound. Regarding the content of acid mucopolysaccharide, from the viewpoint of further improving the effect according to the present invention, the total content of acid mucopolysaccharide is usually from 0.00001 to 5 w/v %, preferably from 0.00005 to 3 w/v %, more preferably from 0.0001 to 2 w/v %, still more preferably from 0.0005 to 1 w/v %, and yet still more preferably from 0.001 to 0.6 w/v %, for example, based on the total amount of the aqueous ophthalmic composition.

In the case of using a hyaluronic acid or a salt thereof as the component (B), in view of more significantly exerting the effect according to the present invention, the content of the hyaluronic acid or the salt thereof is usually from 0.00001 to 1 w/v %, preferably from 0.00005 to 0.5 w/v %, more preferably from 0.0001 to 0.1 w/v %, still more preferably from 0.0005 to 0.05 w/v %, and yet still more preferably from 0.001 to 0.02 w/v %, for example, based on the total amount of the aqueous ophthalmic composition.

In the case of using chondroitin sulfate or a salt thereof as the component (B), in view of more significantly exerting the effect according to the present invention, the content of chondroitin sulfate or a salt thereof is usually from 0.0001 to 5 w/v %, preferably from 0.005 to 3 w/v %, and more preferably from 0.05 to 1 w/v %, for example, based on the total amount of the aqueous ophthalmic composition.

In the case of using an alginic acid or a salt thereof as the component (B), in view of more significantly exerting the effect according to the present invention, the content of the alginic acid or the salt thereof is usually from 0.0001 to 3 w/v %, preferably from 0.001 to 1 w/v %, and more preferably from 0.01 to 0.1 w/v %, for example, based on the total amount of the aqueous ophthalmic composition.

Examples of dextran include dextran 40 and dextran 70. Particularly, dextran 70 is preferred.

In the case of using dextran as the component (B), in view of more significantly exerting the effect according to the present invention, the content of dextran is usually from 0.0001 to 1 w/v %, preferably from 0.001 to 0.1 w/v %, and more preferably from 0.005 to 0.05 w/v %, for example, based on the total amount of the aqueous ophthalmic composition.

In the case of using gellant gum as the component (B), in view of more significantly exerting the effect according to the present invention, the content of gellant gum is usually from 0.0001 to 1 w/v %, preferably from 0.001 to 0.6 w/v %, and more preferably from 0.005 to 0.1 w/v %, for example, based on the total amount of the aqueous ophthalmic composition.

In the case of using glucose as the component (B), in view of more significantly exerting the effect according to the present invention, the content of glucose is usually from 0.0001 to 3 w/v %, preferably from 0.005 to 0.5 w/v %, and more preferably from 0.01 to 0.1 w/v %, for example, based on the total amount of the aqueous ophthalmic composition.

Examples of amino acids and salts thereof include monoamino monocarboxylic acids such as glycine, alanine, γ-aminobutyric acid, and γ-aminovaleric acid; monoamino dicarboxylic acids, such as aspartic acid and glutamic acid, and salts thereof; diaminomonocarboxylic acids, such as arginine and lysine, and salts thereof; and derivatives, such as aminoethylsulfonic acid (taurine), and salts thereof. Commercially available amino acids and salts thereof can also be used. The amino acid and salt thereof may be in any of the L-form, D-form, or DL-form, and examples thereof include a mixture of an equal amount of potassium L-aspartate, magnesium L-aspartate, and potassium magnesium L-aspartate.

Among the amino acids, an aspartic acid, an aminoethylsulfonic acid, and salts thereof are preferred, a mixture of an equal amount of potassium L-aspartate, magnesium L-aspartate, and potassium magnesium L-aspartate, and an aminoethylsulfonic acid are more preferred from the viewpoint of further improving the effect according to the present invention.

The amino acids may be used singly, or in combination of two or more kinds thereof.

The content of the amino acid and salt thereof in the aqueous ophthalmic composition according to the present embodiment is appropriately set depending on the kind and molecular weight of the compound. Regarding the content of the amino acid and salt thereof, from the viewpoint of further improving the effect according to the present invention, the total content of the amino acid and salt thereof is usually from 0.0001 to 3 w/v %, preferably from 0.001 to 2 w/v %, and more preferably from 0.01 to 1 w/v %, for example, based on the total amount of the aqueous ophthalmic composition.

Examples of polyalcohol include straight-chain alcohol (such as glycerin, propylene glycol, ethylene glycol, diethylene glycol, and polyethylene glycol (300, 400, 4000, 6000)) or lactose and sugar alcohol (such as maltose, fructose, sorbitol, maltitol, mannitol, xylitol, and trehalose). The polyalcohol may be synthesized by any known method before use or commercially available polyalcohols may be used. The polyalcohol may be in the D-form or L-form.

Among the polyalcohols, straight-chain alcohol and sugar alcohol are preferred, glycerin, propylene glycol, polyethylene glycol, and mannitol are more preferred, and glycerin, propylene glycol, polyethylene glycol, and D-mannitol are yet still more preferred from the viewpoint of further improving the effect according to the present invention. Further, in view of more significantly exerting the effect according to the present invention, polyethylene glycol is preferably polyethylene glycol 400 or polyethylene glycol 4000.

Such polyalcohol may be used singly, or in combination of two or more kinds thereof.

The content of the polyalcohol in the aqueous ophthalmic composition according to the present embodiment is appropriately set depending on the kind and molecular weight of the compound. Regarding the content of polyalcohol, from the viewpoint of further improving the effect according to the present invention, the total content of polyalcohol is usually from 0.00005 to 8 w/v %, preferably from 0.0001 to 5 w/v %, and more preferably from 0.005 to 4 w/v %, for example, based on the total amount of the aqueous ophthalmic composition.

In the case of using polyethylene glycol as the component (B), in view of more significantly exerting the effect according to the present invention, the content of polyethylene glycol is usually from 0.0001 to 8 w/v %, preferably from 0.001 to 5 w/v %, and more preferably from 0.01 to 1 w/v %, for example, based on the total amount of the aqueous ophthalmic composition.

In the case of using propylene glycol as the component (B), in view of more significantly exerting the effect according to the present invention, the content of propylene glycol is usually from 0.0001 to 8 w/v %, preferably from 0.001 to 5 w/v %, and more preferably from 0.01 to 1 w/v %, for example, based on the total amount of the aqueous ophthalmic composition.

In the case of using D-mannitol as the component (B), in view of more significantly exerting the effect according to the present invention, the content of D-mannitol is usually from 0.0001 to 8 w/v %, preferably from 0.001 to 5 w/v %, and more preferably from 0.01 to 3.5 w/v %, for example, based on the total amount of the aqueous ophthalmic composition.

In the case of using glycerin as the component (B), in view of more significantly exerting the effect according to the present invention, the content of glycerin is usually from 0.0001 to 5 w/v %, preferably from 0.001 to 3 w/v %, and more preferably from 0.01 to 1 w/v %, for example, based on the total amount of the aqueous ophthalmic composition.

Examples of preservatives include hydrochloric-acid alkyldiaminoethylglycine, sodium benzoate, ethanol, benzalkonium chloride, benzethonium chloride, chlorhexidine glyconate, chlorobutanol, sorbic acid, potassium sorbate, sodium dehydroacetate, methyl parahydroxybenzoate, ethyl parahydroxybenzoate, propyl parahydroxybenzoate, butyl parahydroxybenzoate, oxyquinoline sulfate, phenethyl alcohol, benzyl alcohol, biguanide compounds (specifically, polyhexamethylene biguanide etc.), and Glokill (manufactured by Rhodia, product name). Commercially available preservatives can also be used.

Among the preservatives, alkyldiaminoethylglycine hydrochloride, benzalkonium chloride, benzethonium chloride, chlorhexidine glyconate, chlorobutanol, sorbic acid, potassium sorbate, sodium dehydroacetate, methyl parahydroxybenzoate, ethyl parahydroxybenzoate, propyl parahydroxybenzoate, butyl parahydroxybenzoate, and a biguanide compound are preferred, alkyldiaminoethylglycine hydrochloride, benzalkonium chloride, chlorhexidine glyconate, chlorobutanol, sorbic acid, potassium sorbate, methyl parahydroxybenzoate, ethyl parahydroxybenzoate, propyl parahydroxybenzoate, butyl parahydroxybenzoate, and a biguanide compound are more preferred, alkyldiaminoethylglycine hydrochloride, benzalkonium chloride, chlorhexidine glyconate, and a biguanide compound are still more preferred, and benzalkonium chloride, chlorhexidine glyconate, and polyhexamethylene biguanide are yet still more preferred, from the viewpoint of further improving the effect according to the present invention.

The preservatives may be used singly, or in combination of two or more kinds thereof.

The content of the preservative in the aqueous ophthalmic composition according to the present embodiment is appropriately set depending on the kind and molecular weight of the compound. Regarding the content of the preservative, from the viewpoint of further improving the effect according to the present invention, the total content of the preservative is usually from 0.0000001 to 0.5 w/v %, preferably from 0.0000001 to 0.2 w/v %, more preferably from 0.000001 to 0.05 w/v %, and still more preferably from 0.00001 to 0.01 w/v %, for example, based on the total amount of the aqueous ophthalmic composition.

In the case of using polyhexamethylene biguanide as the component (B), in view of more significantly exerting the effect according to the present invention, the content of polyhexamethylene biguanide is usually from 0.0000001 to 0.001 w/v %, preferably from 0.000001 to 0.0005 w/v %, and more preferably from 0.00001 to 0.0001 w/v %, for example, based on the total amount of the aqueous ophthalmic composition.

In the case of using benzalkonium chloride as the component (B), in view of more significantly exerting the effect according to the present invention, the content of benzalkonium chloride is usually from 0.00001 to 0.2 w/v %, preferably from 0.0001 to 0.05 w/v %, and more preferably from 0.001 to 0.01 w/v %, for example, based on the total amount of the aqueous ophthalmic composition.

In the case of using chlorhexidine glyconate as the component (B), in view of more significantly exerting the effect according to the present invention, the content of chlorhexidine glyconate is usually from 0.00001 to 0.2 w/v %, preferably from 0.0001 to 0.05 w/v %, and more preferably from 0.001 to 0.01 w/v %, for example, based on the total amount of the aqueous ophthalmic composition.

Examples of sulfa drugs include sulfisomezole or a salt thereof. Commercially available sulfa drugs can also be used.

Among the sulfa drugs, sulfisomezole and sulfamethoxazole sodium are preferred, and sulfamethoxazole sodium is more preferred from the viewpoint of further improving the effect according to the present invention.

The sulfa drugs may be used singly, or in combination of two or more kinds thereof.

The content of the sulfa drug in the aqueous ophthalmic composition according to the present embodiment is appropriately set depending on the kind and molecular weight of the compound. Regarding the content of the sulfa drug, from the viewpoint of further improving the effect according to the present invention, the total content of the sulfa drug is usually from 0.01 to 6 w/v %, preferably from 0.1 to 5 w/v %, and more preferably from 1 to 4 w/v %, for example, based on the total amount of the aqueous ophthalmic composition.

Examples of vitamins include water soluble vitamins such as vitamin B1, vitamin B2 (flavine adenine dinucleotide), niacin (nicotinic acid and nicotinamide), pantothenic acid, panthenol, vitamin B6 (pyridoxine, pyridoxalisol, and pyridoxamine), biotin, folic acid, and vitamin B12 (cyanocobalamin, hydroxocobalamin, methylcobalamin, and adenosylcobalamin), and salts thereof; and lipid soluble vitamins such as vitamin E (d-α-tocopherol, dl-α-tocopherol, β-tocopherol, γ-tocopherol, δ-tocopherol, etc.), vitamin A (retinal, retinol, retinoic acid, carotene, dehydroretinal, lycopene, etc.), and derivatives thereof. The vitamins may be in the salt form and examples thereof include glavin adenin dinucleotide sodium, pyridoxine hydrochloride, calcium pantothenate, and sodium pantothenate. Further, the vitamins may be derivatives, and examples thereof include tocopherol acetate, retinol acetate, and retinol palmitate. Commercially available vitamins can also be used.

Among the vitamins, flavin adenin dinucleotide sodium, panthenol, sodium pantothenate, calcium pantothenate, pyridoxine hydrochloride, cyanocobalamin, retinol acetate, retinol palmitate, and tocopherol acetate are preferred, and pyridoxine hydrochloride, cyanocobalamin, retinol palmitate, and tocopherol acetate are particularly preferred from the viewpoint of further improving the effect according to the present invention.

From another viewpoint, a lipid soluble vitamin is preferred, retinol, tocopherol or derivatives thereof are more preferred, retinol acetate, retinol palmitate, and tocopherol acetate are more preferred, and retinol palmitate and tocopherol acetate are yet still more preferred.

The vitamins may be used singly, or in combination of two or more kinds thereof.

The content of vitamin in the aqueous ophthalmic composition according to the present embodiment is appropriately set depending on the kind and molecular weight of the compound. Regarding the content of vitamin, from the viewpoint of further improving the effect according to the present invention, the total content of vitamin is usually from 0.00001 to 1 w/v %, preferably from 0.00005 to 0.5%, and more preferably from 0.0001 to 0.1 w/v %, for example, based on the total amount of the aqueous ophthalmic composition.

In the case of using a water-soluble vitamin as the component (B), regarding the content of the water soluble vitamin, the total content of the water soluble vitamin is usually from 0.0001 to 1 w/v %, preferably from 0.0002 to 0.5 w/v %, more preferably from 0.0005 to 0.1 w/v %, and still more preferably from 0.001 to 0.1 w/v % based on the total amount of the aqueous ophthalmic composition.

In the case of using pyridoxine hydrochloride as the component (B), regarding the content of pyridoxine hydrochloride, for example, the total content of pyridoxine hydrochloride is usually from 0.0001 to 1 w/v %, preferably from 0.0005 to 0.1 w/v %, and more preferably from 0.001 to 0.1 w/v % based on the total amount of the aqueous ophthalmic composition.

In the case of using a lipid soluble vitamin as the component (B), regarding the content of the lipid soluble vitamin, for example, the total content of the lipid soluble vitamin is usually from 0.00001 to 0.5 w/v %, preferably from 0.00005 to 0.15 w/v %, and more preferably from 0.0005 to 0.05 w/v % based on the total amount of the aqueous ophthalmic composition.

In the case of using tocopherol as the component (B), regarding the content of tocopherol, for example, the total content of tocopherol is usually from 0.0001 to 0.5 w/v %, preferably from 0.0005 to 0.15 w/v %, and more preferably from 0.0005 to 0.05 w/v % based on the total amount of the aqueous ophthalmic composition.

In the case of using retinol as the component (B), regarding the content of retinol, for example, the total content of retinol is usually from 1 to 200,000 I.U./100 mL, preferably from 500 to 100,000 I.U./100 mL, and more preferably from 5000 to 50,000 I.U./100 mL based on the total amount of the aqueous ophthalmic composition.

Vitamin A is, for example, retinol palmitate manufactured by DSM (I.U. vitamin A1=0.550 μg). Note that I.U. is an international unit for the method described in the vitamin A assay of the Japanese Pharmacopoeia, 16th version.

In the case of using cyanocobalamin as the component (B), in view of more significantly exerting the effect according to the present invention, the content of cyanocobalamin is usually from 0.0001 to 0.1 w/v %, preferably from 0.0002 to 0.05 w/v %, and more preferably from 0.001 to 0.02 w/v %, for example, based on the total amount of the aqueous ophthalmic composition.

Examples of inorganic salts include metal chlorides such as calcium chloride, magnesium chloride, sodium chloride, potassium chloride, ammonium chloride, and zinc chloride; and metal sulfates such as calcium sulfate, magnesium sulfate, sodium sulfate, potassium sulfate, and ammonium sulfate. Sodium chloride, potassium chloride, calcium chloride, magnesium sulfate, and zinc chloride are preferred. Commercially available inorganic salts can also be used.

Among the inorganic salts, metal chloride and metal sulfate are preferred, sodium chloride, potassium chloride, calcium chloride, zinc chloride, and magnesium sulfate are preferred, and sodium chloride, zinc chloride and magnesium sulfate are particularly preferred from the viewpoint of further improving the effect according to the present invention.

The inorganic salts may be used singly, or in combination of two or more kinds thereof.

The content of the inorganic salt in the aqueous ophthalmic composition according to the present embodiment is appropriately set depending on the kind and molecular weight of the compound. Regarding the content of the inorganic salt, from the viewpoint of further improving the effect according to the present invention, the total content of the inorganic salt is usually from 0.00001 to 8 w/v %, preferably from 0.0001 to 5 w/v %, and more preferably from 0.0001 to 1 w/v %, for example, based on the total amount of the aqueous ophthalmic composition.

In the case of using calcium chloride, magnesium chloride, sodium chloride, potassium chloride, ammonium chloride, calcium sulfate, magnesium sulfate, sodium sulfate, potassium sulfate, and ammonium sulfate as the component (B), it is usually from 0.0001 to 8 w/v %, preferably from 0.0005 to 5 w/v %, and more preferably from 0.001 to 1 w/v %.

In the case of using zinc chloride as the component (B), it is usually from 0.00001 to 0.1 w/v %, preferably from 0.0001 to 0.03 w/v %, and more preferably from 0.0001 to 0.003 w/v %.

Examples of ocular muscle modulator components include cholinesterase inhibitors having an active center similar to acetylcholine, and specific examples thereof include neostigmine methylsulfate, tropicamide, helenien, and atropine sulfate. Commercially available ocular muscle modulator components can also be used.

Among the ocular muscle modulator components, neostigmine or a salt thereof is preferred, and neostigmine methylsulfate is more preferred from the viewpoint of further improving the effect according to the present invention.

The ocular muscle modulator components may be used singly, or in combination of two or more kinds thereof.

The content of the ocular muscle modulator component in the aqueous ophthalmic composition according to the present embodiment is appropriately set depending on the kind and molecular weight of the compound. Regarding the content of the ocular muscle modulator component, from the viewpoint of further improving the effect according to the present invention, the total content of the ocular muscle modulator component is usually from 0.0001 to 0.05 w/v %, preferably from 0.0005 to 0.01 w/v %, and more preferably from 0.001 to 0.005 w/v %, for example, based on the total amount of the aqueous ophthalmic composition.

Examples of vasoconstrictors (decongestants) include tetryzoline, naphazoline, epinephrine, ephedrine, methylephedrine, phenylephrine, and salts thereof. Commercially available vasoconstrictors can also be used.

Among the vasoconstrictors, tetryzoline and naphazoline or salts thereof are preferred, tetrahydrozoline hydrochloride, naphazoline hydrochloride, and naphazoline nitrate are more preferred, and tetrahydrozoline hydrochloride and naphazoline hydrochloride are yet still more preferred, and naphazoline hydrochloride is particularly preferred from the viewpoint of further improving the effect according to the present invention.

The vasoconstrictors (decongestants) may be used singly, or in combination of two or more kinds thereof.

The content of the vasoconstrictor in the aqueous ophthalmic composition according to the present embodiment is appropriately set depending on the kind and molecular weight of the compound. Regarding the content of the vasoconstrictor, from the viewpoint of further improving the effect according to the present invention, the total content of the vasoconstrictor is usually from 0.0001 to 0.1 w/v %, preferably from 0.0005 to 0.05 w/v %, and more preferably from 0.0005 to 0.005 w/v %, for example, based on the total amount of the aqueous ophthalmic composition.

Examples of stabilizers include dibutylhydroxytoluene (BHT), trometamol, sodium formaldehyde sulfoxylate (Rongalite), sodium pyrosulfife, sodium sulfite, sodium bisulfite, sodium thiosulfate, monoethanolamine, aluminium monostearate, and glyceryl monostearate. Commercially available stabilizers can also be used.

Among the stabilizers, dibutylhydroxytoluene, trometamol, and monoethanolamine are preferred, and dibutylhydroxytoluene is more preferred from the viewpoint of further improving the effect according to the present invention.

The stabilizers may be used singly, or in combination of two or more kinds thereof.

The content of the stabilizer in the aqueous ophthalmic composition according to the present embodiment is appropriately set depending on the kind and molecular weight of the compound. Regarding the content of the stabilizer, from the viewpoint of further improving the effect according to the present invention, for example, the total content of the stabilizer is usually from 0.000001 to 3 w/v %, preferably from 0.000001 to 2 w/v %, more preferably from 0.00001 to 1.5 w/v %, still more preferably from 0.0001 to 1 w/v %, and yet still more preferably from 0.0001 to 0.05 w/v % based on the total amount of the aqueous ophthalmic composition.

In the case of using dibutylhydroxytoluene as the component (B), in view of more significantly exerting the effect according to the present invention, the content of dibutylhydroxytoluene is usually from 0.000001 to 0.1 w/v %, preferably from 0.00001 to 0.01 w/v %, and more preferably from 0.0001 to 0.005 w/v %, for example, based on the total amount of the aqueous ophthalmic composition.

In the case of using trometamol as the component (B), in view of more significantly exerting the effect according to the present invention, the content of trometamol is usually from 0.0001 to 3 w/v %, preferably from 0.001 to 2 w/v %, and more preferably from 0.01 to 1 w/v %, for example, based on the total amount of the aqueous ophthalmic composition.

In the case of using monoethanolamine as the component (B), in view of more significantly exerting the effect according to the present invention, the content of monoethanolamine is usually from 0.0001 to 2 w/v %, preferably from 0.001 to 1.5 w/v %, and more preferably from 0.01 to 0.5 w/v %, for example, based on the total amount of the aqueous ophthalmic composition.

Examples of polyoxyethylene polyoxypropylene glycol include POE (196) POP (67) glycol (Poloxamer 407, Pluronic F-127), POE (200) POP (70) glycol, POE (120) POP (40) glycol (Pluronic F-87), POE (160) POP (30) glycol (Pluronic F-68, Poloxamer-188), polyoxyethylene (54) polyoxypropylene (39) glycol (Pluronic P-85, Poloxamer 235), polyoxyethylene (20) polyoxypropylene (20) glycol (Pluronic L-44, Poloxamer 124), polyoxyethylene (42) polyoxypropylene (67) glycol (Pluronic P123, Poloxamer 403). Commercially available polyoxyethylene polyoxypropylene glycol can also be used.

Among polyoxyethylene polyoxypropylene glycol, POE (160) POP (30) glycol (Pluronic F-68, Poloxamer 188) and POE (196) POP (67) glycol (Poloxamer 407, Pluronic F127) are preferred.

Polyoxyethylene polyoxypropylene glycol may be used singly, or in combination of two or more kinds thereof.

The content of polyoxyethylene polyoxypropylene glycol in the aqueous ophthalmic composition according to the present embodiment is appropriately set depending on the kind and molecular weight of the compound. Regarding the content of polyoxyethylene polyoxypropylene glycol, from the viewpoint of further improving the effect according to the present invention, the total content of polyoxyethylene polyoxypropylene glycol is usually from 0.00001 to 10 w/v %, preferably from 0.0001 to 8 w/v %, more preferably from 0.001 to 5 w/v %, and still more preferably from 0.01 to 0.1 w/v %, for example, based on the total amount of the aqueous ophthalmic composition.

Examples of vegetable oils include sesame oil, castor oil, olive oil, soybean oil, peanut oil, almond oil, wheat germ oil, camellia oil, maize oil, rapeseed oil, sunflower oil, cottonseed oil, and coconut oil. Commercially available vegetable oils may be used.

Among the vegetable oils, sesame oil and castor oil are preferred, and sesame oil is more preferred from the viewpoint of further improving the effect according to the present invention.

The vegetable oils may be used singly, or in combination of two or more kinds thereof.

The content of the vegetable oil in the aqueous ophthalmic composition according to the present embodiment is appropriately set depending on the kind and molecular weight of the compound. Regarding the content of the vegetable oil, from the viewpoint of further improving the effect according to the present invention, the total content of the vegetable oil is usually from 0.00001 to 3 w/v %, preferably from 0.0001 to 1 w/v %, and more preferably from 0.001 to 0.1 w/v % for example, based on the total amount of the aqueous ophthalmic composition.

Preferably, the aqueous ophthalmic composition according to the present embodiment further contains (C) a nonionic surfactant (simply referred to as “component (C)”). The effect according to the present invention is more significantly exerted by containing the component (C).

(C) The nonionic surfactant is not particularly limited as long as it is a medically, pharmacologically (pharmaceutically) or physiologically acceptable nonionic surfactant. Specific examples of (C) nonionic surfactants include POE sorbitan fatty acid esters such as monolaurate POE (20) sorbitan (polysorbate 20), monooleate POE (20) sorbitan (polysorbate 80), POE sorbitan monostearate (polysorbate 60), POE sorbitan tristearate (polysorbate 65); POE hydrogenated castor oils such as POE hydrogenated castor oil 5, POE hydrogenated castor oil 10, POE hydrogenated castor oil 20, POE hydrogenated castor oil 40, POE hydrogenated castor oil 50, POE hydrogenated castor oil 60, POE hydrogenated castor oil 100; POE castor oils such as POE castor oil 3, POE castor oil 4, POE castor oil 6, POE castor oil 7, POE castor oil 10, POE castor oil 13.5, POE castor oil 17, POE castor oil 20, POE castor oil 25, POE castor oil 30, POE castor oil 35, and POE castor oil 50; POE-POP block copolymer adducts of ethylenediamine such as polyethylene glycol monostearate (2E.O.), polyethylene glycol monostearate (4E.O.), polyethylene glycol monostearate (9E.O.), polyethylene glycol monostearate (10E.O.), polyethylene glycol monostearate (23E.O.), polyethylene glycol monostearate (25E.O.), polyethylene glycol monostearate (32E.O.), polyethylene glycol monostearate (40E.O.) polyoxyl 40 stearate, polyethylene glycol monostearate (45E.O.), polyethylene glycol monostearate (55E.O.), polyethylene glycol monostearate (75E.O.), and polyethylene glycol monostearate (140E.O.), polyethylene glycol monostearate and poloxamine; POE alkyl ether such as POE (9) lauryl ether; POE-POP alkyl ethers such as POE (20) POP (4) cetyl ether; POE alkylphenyl ethers such as POE (10) nonylphenyl ether; and tyloxapol. Note that the numbers in brackets are addition mol numbers.

More preferably, the aqueous ophthalmic composition according to the present embodiment contains (C) two or more kinds of nonionic surfactants. The combination of (C) the nonionic surfactants is not particularly limited. From the viewpoint of more significantly exerting the effect according to the present invention, it is preferable to combine at least one of (C-1) the nonionic surfactants having an HLB value of 10 or more, (simply referred to as “component (C-1)”) and at least one of (C-2) the nonionic surfactants having an HLB value of less than 10 (simply referred to as “component (C-2)”). In this case, the component (C-1) has preferably an HLB value of 11 or more, and more preferably an HLB value of 13 or more. The component (C-2) has preferably an HLB value of 8 or less and more preferably an HLB value of 6 or less.

Examples of component (C-1) include POE sorbitan fatty acid esters such as monolaurate POE (20) sorbitan (polysorbate 20), monooleate POE (20) sorbitan (polysorbate 80), POE sorbitan monostearate (polysorbate 60), and POE sorbitan tristearate (polysorbate 65); POE hydrogenated castor oils having an average addition mole number of ethylene oxide of 20 or more such as POE hydrogenated castor oil 20, POE hydrogenated castor oil 40, POE hydrogenated castor oil 50, POE hydrogenated castor oil 60, and POE hydrogenated castor oil 100; POE castor oils having an average addition mole number of ethylene oxide of 23 or more such as POE castor oil 25, POE castor oil 30, POE castor oil 35, and POE castor oil 50; polyethylene glycol monostearate having an average addition mole number of ethylene oxide of 7 or more such as polyethylene glycol monostearate (9E.O.), polyethylene glycol monostearate (10E.O.), polyethylene glycol monostearate (23E.O.), polyethylene glycol monostearate (25E.O.), polyethylene glycol monostearate (32E.O.), polyethylene glycol monostearate (40E.O., polyoxyl 40 stearate), polyethylene glycol monostearate (45E.O.), polyethylene glycol monostearate (55E.O.), polyethylene glycol monostearate (75E.O.), polyethylene glycol monostearate (140E.O.); poloxamine, POE (20) POP (4) cetyl ether, and POE (10) nonylphenyl ether. Among them, monooleate POE (20) sorbitan (polysorbate 80), POE hydrogenated castor oil 40, POE hydrogenated castor oil 60, POE castor oil 35, and polyoxyl 40 stearate are preferred, and monooleate POE (20) sorbitan (polysorbate 80) and POE hydrogenated castor oil 60 are more preferred from the viewpoint of more significantly exerting the effect according to the present invention.

Examples of component (C-2) include POE hydrogenated castor oils having an average addition mole number of ethylene oxide of less than 20 such as POE hydrogenated castor oil 5 and POE hydrogenated castor oil 10; POE castor oil having an average addition mole number of ethylene oxide of less than 23 such as POE castor oil 3, POE castor oil 4, POE castor oil 6, POE castor oil 7, POE castor oil 10, POE castor oil 13.5, POE castor oil 17, and POE castor oil 20; and polyethylene glycol monostearate having an average addition mole number of ethylene oxide of less than 7 such as polyethylene glycol monostearate (2E.O.) and polyethylene glycol monostearate (4E.O.). Among them, POE hydrogenated castor oil having an average addition mole number of ethylene oxide of less than 20 and POE castor oil having an average addition mole number of ethylene oxide of less than 23 are preferred, POE castor oil having an average addition mole number of ethylene oxide of less than 23 is more preferred, POE castor oil 3 and POE castor oil 10 are still more preferred, and POE castor oil 3 is particularly preferred from the viewpoint of more significantly exerting the effect according to the present invention.

In the case of using the component (C), the content of the component (C) in the aqueous ophthalmic composition according to the present embodiment is not particularly limited and is appropriately set depending on the use and dosage form of the aqueous ophthalmic composition. Regarding the lower limit of the content of the component (C), from the viewpoint of more significantly exerting the effect according to the present invention, the total content of the component (C) may be 0.0001 w/v % or more, preferably 0.001 w/v % or more, more preferably 0.01 w/v % or more, and still more preferably 0.1 w/v % or more, for example, based on the total amount of the aqueous ophthalmic composition. Although the upper limit of the content of the component (C) is not limited, the total content of the component (C) may be 20 w/v % or less, preferably 10 w/v % or less, more preferably 5 w/v % or less, still more preferably 1 w/v % or less, and particularly preferably 0.5 w/v % or less, for example, based on the total amount of the aqueous ophthalmic composition.

Regarding a specific range of the content of the component (C), from the viewpoint of more significantly exerting the effect according to the present invention, the total content of the component (C) may be from 0.0001 to 20 w/v %, preferably from 0.001 to 10 w/v %, more preferably from 0.01 to 5 w/v %, and still more preferably from 0.1 to 1 w/v %, for example, based on the total amount of the aqueous ophthalmic composition.

In the case of combining the component (C-1) and the component (C-2), from the viewpoint of more significantly exerting the effect according to the present invention, the content of the component (C-1) and the content of the component (C-2) are exemplified as follows:

The component (C-1): the total content of the component (C-1) may be from 0.0001 to 10 w/v %, preferably from 0.001 to 7 w/v %, more preferably from 0.01 to 4 w/v %, still more preferably from 0.01 to 1 w/v %, and particularly preferably from 0.01 to 0.5% based on the total amount of the aqueous ophthalmic composition.

The component (C-2): the total content of the component (C-2) may be from 0.0001 to 10 w/v %, preferably from 0.001 to 8 w/v %, more preferably from 0.005 to 5 w/v %, and still more preferably from 0.01 to 0.5 w/v % based on the total amount of the aqueous ophthalmic composition.

Regarding the ratio of the component (A) and the component (C) in the aqueous ophthalmic composition according to the present embodiment, from the viewpoint of more significantly exerting the effect according to the present invention, the total amount of the component (C) may be from 0.0001 to 1000000 parts by weight, preferably from 0.001 to 100000 parts by weight, more preferably from 0.001 to 50000 parts by weight, and still more preferably from 0.01 to 10000 parts by weight, for example, based on 1 part by weight of the total amount of the component (A).

In the case of combining the component (C-1) and the component (C-2), from the viewpoint of more significantly exerting the effect according to the present invention, the ratio of the component (A) and the component (C) are exemplified as follows:

Regarding the ratio of the component (C-1) and the component (C-2) in the aqueous ophthalmic composition according to the present embodiment, the total amount of the component (C-2) may be from 0.001 to 1000 parts by weight preferably from 0.01 to 100 parts by weight, and more preferably from 0.1 to 10 parts by weight, for example, based on 1 part by weight of the total amount of the component (C-1).

Regarding the ratio of the component (A) and the component (C-1) in the aqueous ophthalmic composition according to the present embodiment, from the viewpoint of more significantly exerting the effect according to the present invention, the total amount of the component (C-1) may be from 0.0001 to 1000000 parts by weight, preferably from 0.001 to 100000 parts by weight, more preferably from 0.001 to 50000 parts by weight, and still more preferably from 0.01 to 10000 parts by weight, for example, based on 1 part by weight of the total amount of the component. (A).

Regarding the ratio of the component (A) and the component (C-2) in the aqueous ophthalmic composition according to the present embodiment, from the viewpoint of more significantly exerting the effect according to the present invention, the total amount of the component (C-2) may be from 0.0001 to 1000000 parts by weight, preferably from 0.001 to 100000 parts by weight, more preferably from 0.001 to 50000 parts by weight, and still more preferably from 0.01 to 10000, for example, based on 1 part by weight of the total amount of the component (A).

Preferably, the aqueous ophthalmic composition according to the present embodiment further contains (D) a buffer (simply referred to as “component (D)”). The effect according to the present invention is more significantly exerted by containing the component (D).

(D) The buffer is not particularly limited as long as it is a medically, pharmacologically (pharmaceutically) or physiologically acceptable buffer. Specific examples of (D) buffers include boric acid buffers, phosphoric acid buffers, carbonic acid buffers, citric acid buffers, and acetic acid buffers. Among these, the boric acid buffers, the phosphoric acid buffers, the carbonic acid buffers, and the citric acid buffers are preferred.

Examples of boric acid buffers include borate salts such as a boric acid, a boric acid alkali metal salt, and a boric acid alkaline earth metal salt. Examples of phosphoric acid buffers include phosphoric salts such as a phosphoric acid, a phosphoric acid alkali metal salt, and a phosphoric acid alkaline earth metal salt. Examples of carbonic acid buffers include carbonates such as a carbonic acid, an alkali carbonate metal salt, and an alkali carbonate earth metal salt. Examples of citric acid buffers include a citric acid, a citric acid alkali metal salt, and a citric acid alkaline earth metal salt. As the boric acid buffer or the phosphoric acid buffer, a hydrate of borate salt or phosphoric salt may be used. More specifically, the boric acid buffer is, for example, a boric acid or a salt thereof (sodium borate, potassium tetraborate, potassium metaborate, ammonium pentaborate, borax, etc.), the phosphoric acid buffer, is, for example, a phosphoric acid or a salt thereof (disodium hydrogenphosphate, sodium dihydrogen phosphate, potassium dihydrogen phosphate, trisodium monophosphate, dipotassium phosphate, calcium monohydrogen phosphate, monobasic calcium phosphate, etc.), the carbonic acid buffer is, for example, a carbonic acid or a salt thereof (sodium hydrogen carbonate, sodium carbonate, ammonium carbonate, potassium carbonate, calcium carbonate, potassium bicarbonate, magnesium carbonate, etc.), the citric acid buffer is, for example, a citric acid or a salt thereof (sodium citrate, potassium citrate, calcium citrate, sodium dihydrogen citrate, disodium citrate, etc.), and the acetic acid buffer is, for example, an acetic acid or a salt thereof (ammonium acetate, potassium acetate, calcium acetate, sodium acetate, etc.). These buffers may be used singly, or in an arbitrary combination of two or more kinds thereof.

Among the buffers, the boric acid buffer and the phosphoric acid buffer are particularly preferred from the viewpoint of more significantly exerting the effect according to the present invention. The boric acid buffer is preferably a combination of a boric acid and a salt thereof, more preferably a combination of a boric acid and an alkali metal salt and/or an alkaline earth metal salt of boric acid, still more preferably, a combination of a boric acid and an alkali metal salt of boric acid, and yet still more preferably a combination of a boric acid and borax. The phosphoric acid buffer may be preferably a combination of primary phosphage and secondary phosphate, more preferably a combination of an alkali metal salt of primary phosphate and an alkali metal salt of secondary phosphate, and still more preferably a combination of sodium dihydrogen phosphate and disodium hydrogenphosphate.

In the case of using the component (D), the content of the component (D) in the aqueous ophthalmic composition according to the present embodiment is not particularly limited and is appropriately set depending on the use and dosage form of the aqueous ophthalmic composition. Regarding the lower limit of the content of the component (D), from the viewpoint of more significantly exerting the effect according to the present invention, the total content of the component (D) may be 0.001 w/v % or more, preferably 0.01 w/v % or more, and more preferably 0.1 w/v % or more, for example, based on the total amount of the aqueous ophthalmic composition. Although the upper limit of the content of the component (D) is not limited, the total content of the component (D) may be 10 w/v % or less, preferably 5 w/v % or less, and more preferably 3 w/v % or less, for example, based on the total amount of the aqueous ophthalmic composition.

Regarding a specific range of the content of the component (D), from the viewpoint of more significantly exerting the effect according to the present invention, the total content of the component (D) may be from 0.001 to 10 w/v %, preferably from 0.01 to 5 w/v %, and more preferably from 0.1 to 3 w/v %, for example, based on the total amount of the aqueous ophthalmic composition.

Regarding the ratio of the component (A) and the component (D) in the aqueous ophthalmic composition according to the present embodiment, from the viewpoint of more significantly exerting the effect according to the present invention, the total amount of the component (D) may be from 0.0001 to 30000 parts by weight, preferably from 0.001 to 30000 parts by weight, and more preferably from 0.01 to 20000 parts by weight, for example, based on 1 part by weight of the total amount of the component (A).

Preferably, the aqueous ophthalmic composition according to the present embodiment further contains (E) a terpenoid compound (simply referred to as “component (E)”). The component (E) is not particularly limited as long as it is a medically, pharmacologically (pharmaceutically) or physiologically acceptable component. Specific examples of component (E) include menthol, camphor, borneol, geraniol, cineole, citronellol, menton, carvone, anethole, eugenol, limonene, linalool, linalyl acetate, and derivatives thereof. The component (E) may be in any one of d-form, l-form and dl-form. Further, as the component (E), essential oils containing the component (E) may be used. Examples of essential oils include eucalyptus oil, bergamot oil, peppermint oil, cool mint oil, spearmint oil, mentha oil, fennel oil, cinnamon oil, and rose oil. These component (E) may be used singly, or in an arbitrary combination of two or more kinds thereof.

Among the component (E), dl-menthol, l-menthol, dl-camphor, d-camphor, d-borneol or geraniol is preferred in view of more significantly exerting the effect according to the present invention. Examples of essential oils including these include cool mint oil, peppermint oil, mentha oil, and camphor oil. As the component (E), dl-menthol, l-menthol, dl-camphor, d-camphor, d-borneol, and geraniol are more preferred, l-menthol, d-camphor, and dl-camphor are still more preferred, and l-menthol is yet still more preferred.

In the case of using the component (E), the content of the component (E) in the aqueous ophthalmic composition according to the present embodiment varies depending on the kind of the component (E) to be used, the kind and amount of another component, and the use and dosage form of the aqueous ophthalmic composition and cannot be uniformly specified. The total content of the component (E) may be from 0.00001 to 10 w/v %, preferably from 0.0001 to 10 w/v %, more preferably from 0.0005 to 5 w/v %, still more preferably from 0.001 to 3 w/v %, and yet still more preferably from 0.001 to 1 w/v %, for example, based on the total amount of the aqueous ophthalmic composition.

The pH of the aqueous ophthalmic composition according to the present embodiment is not particularly limited as long as it is within a medically, pharmacologically (pharmaceutically) or physiologically acceptable range. The pH of the aqueous ophthalmic composition according to the present embodiment may be, for example, from 4.0 to 9.5, preferably from 4.0 to 9.0, more preferably from 4.5 to 9.0, still more preferably from 4.5 to 8.5, and yet still more preferably from 5.0 to 8.5.

If necessary, the aqueous ophthalmic composition according to the present embodiment can be adjusted to an osmotic pressure ratio within the range that is acceptable to biological bodies. The appropriate osmotic pressure ratio varies depending on the application site and dosage form or the like and is, for example, from 0.4 to 5.0, preferably from 0.6 to 3.0, and more preferably from 0.8 to 2.0. The osmotic pressure can be adjusted by known methods in the art using organic salt, polyalcohol or the like. The osmotic pressure ratio is a ratio of an osmotic pressure of a sample to 286 mOsm (osmotic pressure of a 0.9 w/v % sodium chloride aqueous solution) based on the Japanese Pharmacopoeia, 16th version, and the osmotic pressure is measured with reference to the osmometry determination described in the Japanese Pharmacopoeia (cryoscopic method). Note that the standard solution for measuring an osmotic pressure ratio (aqueous 0.9 w/v % sodium chloride solution) is prepared by drying sodium chloride (The Japanese Pharmacopeia standard reagent) at 500 to 650° C. for 40 to 50 minutes, and thereafter allowing it to cool in a desiccator (silica gel), accurately weighing 0.900 g of the cooled sample, and dissolving the sample in purified water to make up a volume of 100 mL, or alternatively, a commercially available standard solution for measuring an osmotic pressure ratio (aqueous 0.9 w/v % sodium chloride solution) is used.

The aqueous ophthalmic composition according to the present embodiment may contain an appropriate amount of a combination of components selected from the above components, various harmacological active components, and physiologically active components as long as the range does not impair the effect of the present invention. The components are not particularly limited, and examples thereof include active components in ophthalmic drugs described in Standards of Production and Sale of OTC Drugs 2012 (supervised by SOCIETY FOR REGULATORY SCIENCE OF MEDICAL PRODUCTS). Specific examples of components used for ophthalmic drugs include the followings:

antihistamine drugs: e.g., iproheptine, diphenhydramine, chlorpheniramine maleate, ketotifen fumarate, olopatadine hydrochloride, and levocabastine hydrochloride;

antiallergic drugs: e.g., disodium cromoglycate, tranilast, and pemirolast potassium;

steroid drugs: e.g., fluticasone propionate, fluticasone furoate, mometasone furoate, beclometasone dipropionate, and flunisolide;

anti-inflammatory drugs: e.g., glycyrrhetinic acid, glicyrrhizic acid, pranoprofen, methyl salicylate, glycol salicylate, allantoin, tranexamic acid, ε-aminocaproic acid, berberine, azulene sodium sulfonate, lysozyme, and licorice;

astringent drugs: e.g., hydrozincite, zinc lactate, and zinc sulfate;

local anesthetics: e.g., lidocaine; and

others: e.g., sulfisoxazole, sulfisomidine, and salts thereof.

To the aqueous ophthalmic composition according to the present embodiment, one or more additives appropriately selected from various additives may be added in an appropriate amount by an ordinary method depending on the use and dosage form, as long as the range does not impair the effect of the present invention. Examples of additives include various additives described in “Iyakuhin Tenkabutu Jiten 2007 (Encyclopedia of Pharmaceutical Excipients), edited by Japan Pharmaceutical Excipients Council Japan”. Examples of typical components include the followings:

carriers: e.g., aqueous solvents such as water and hydrous ethanol;

chelating drugs: e.g., ethylenediamine diacetate (EDDA), ethylenediamine triacetic acid, ethylenediaminetetraacetate (edetic acid, EDTA), N-(2-Hydroxyethyl)ethylenediamine-triacetic acid (HEDTA), and diethylenetriaminepentaacetic acid (DTPA); and

bases: e.g., octyl dodecanol, titanium oxide, potassium bromide, Plastibase, liquid paraffin, light liquid paraffin, and purified lanolin.

Note that it is preferable that the aqueous ophthalmic composition according to the present embodiment substantially does not contain lecithin as an additive, and it is more preferable that it does not contain lecithin. Thus, the aqueous ophthalmic composition has no unpleasant odor peculiar to lecithin, thereby more significantly exerting the effect according to the present invention.

Water to be used for the aqueous ophthalmic composition according to the present embodiment may be medically, pharmacologically (pharmaceutically) or physiologically acceptable water. Examples of such water include distilled water, water, purified water, sterile purified water, water for injection, and distilled water for injection. These definitions are based on the Japanese Pharmacopoeia, 16th version.

The aqueous ophthalmic composition according to the present embodiment can be prepared by adding desired amounts of the component (A), the component (B), and other components, if necessary and mixing them so as to have a desired concentration. For example, it can be prepared by dissolving or dispersing these components in purified water so as to be adjusted to a predetermined pH and osmotic pressure, and sterilizing the resulting mixture by filter sterilization or the like. Regarding the dissolution of the component (A) and other highly hydrophobic components, it may be performed by previously mixing the component (C) and the component (D) or a component having a solubilization effect and stirring the mixture, and further adding purified water.

The aqueous ophthalmic composition according to the present embodiment may be in various dosage forms depending on the intended use. Examples of dosage forms include liquid drugs, gel drugs, and semi solid drugs (ointment etc.). The aqueous ophthalmic composition according to the present embodiment is preferably a liquid drug.

The aqueous ophthalmic composition according to the present embodiment may be used as, for example, eye drops (also referred to as ophthalmic solutions or ophthalmic drugs, and further examples of the eye drops include eye drops that can be instilled into eyes during use of contact lenses), artificial tears, eye washes (also referred to as collyriums or eye lotions, and further examples of the eye washes include eye washes that can wash eyes during use of contact lenses), solutions for wearing a contact lens and contact lens care agents (including contact lens disinfecting solutions, contact lens storage solutions, contact lens cleaning solutions, contact lens cleaning and storage solutions, and the like). Note that the term “contact lens” includes hard contact lenses and soft contact lenses (ionic and non-ionic lenses are included and silicone hydro gel contact lenses and non-silicone hydro-gel contact lenses are included).

Among them, the eye drops can be simply used. Accordingly, in the case of feeling of ocular discomfort when blinking, it is possible to put drops of the eye drops into eyes within the acceptable use range. In view of this effect, the aqueous ophthalmic composition according to the present embodiment can be preferably used as eye drops.

The aqueous ophthalmic composition according to the present embodiment is provided after being contained in an arbitrary container. The container for containing the aqueous ophthalmic composition according to the present embodiment is not particularly limited and may be made of glass, for example, or may be made of plastic. Preferably, it is made of plastic. Examples of plastics include polyethylene terephthalate, polyalylate, polyethylenenaphthalate, polycarbonate, polyethylene, polypropylene, polyimide, copolymers of monomers forming these, and mixtures of two or more kinds thereof. Polyethylene terephthalate is preferred. Further, the container for containing the aqueous ophthalmic composition according to the present embodiment may be a transparent container whose inside can be visually recognized or may be an opaque container whose inside is hard to be visually recognized. The transparent container is preferred. Here, the term “transparent container” includes both a transparent and colorless container and a colored transparent container.

The container may be equipped with a nozzle. The material of the nozzle is not particularly limited, and may be made of, for example, glass or plastic. Preferably, it is made of plastic. Examples of plastics include polybutylene terephthalate, polyethylene, polypropylene, copolymers of monomers forming these, and mixtures of two or more kinds thereof. Polyethylene is preferred.

In the aqueous ophthalmic composition according to the present embodiment, the vaseline may be emulsified or dissolved. In the aqueous ophthalmic composition according to the present embodiment, it is preferable that the vaseline is not separated. Note that the wording “is not separated” indicates that in the case of being allowed to stand for 1 hour, separation between an oil phase containing vaseline and an aqueous phase does not occur. Further, the aqueous ophthalmic composition according to the present embodiment preferably exhibits high light transmittance. Therefore, in order to visually confirm whether the composition is a drug product having high light transmittance, a transparent container is preferably used as a container for accommodating the aqueous ophthalmic composition according to the present embodiment. The drug product has high light transmittance and is contained in the transparent container, whereby Foreign Matter Test or the like can be easily performed with naked eyes. Thus, regarding the usage form of the aqueous ophthalmic composition of the present invention, an ophthalmic solution and an eyewash for which the Foreign Matter Test is required are more preferred, and the ophthalmic solution is particularly preferred.

Regarding the light transmittance of the aqueous ophthalmic composition according to the present embodiment, the light transmittance at a wavelength of 660 nm may be, for example, 70% or more, preferably 75% or more, more preferably 80% or more, still more preferably 85% or more, yet still more preferably 90% or more, and particularly preferably 95% or more.

In the aqueous ophthalmic composition according to the present embodiment, the dynamic contact angle is reduced, thereby increasing the fluidity of the liquid. Consequently, the effect of improving smooth blinking, the effect of improving clear vision (effect of improving blurred vision), the effect of preventing infectious diseases, and the effect of improving visual function are expected.

[2. Suppression of Ocular Discomfort]

In the aqueous ophthalmic composition according to the present embodiment, the dynamic contact angle (angle of advance) is improved and the fluidity of the liquid on the ocular surface is increased. For example, during motion such as blinking or tear exchange, wetting is easily spread to the solid such as eyelid, cornea or contact lens. Thus, the aqueous ophthalmic composition according to the present embodiment produces an effect of reducing ocular discomfort, particularly discomfort during use of contact lenses.

Therefore, as one embodiment of the present invention, there is provided an agent for reducing ocular discomfort consisting of the aqueous ophthalmic composition containing (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil (preferably, a discomfort-reducing agent during use of contact lenses). As one embodiment of the present invention, there is provided an aqueous ophthalmic composition for use in reducing ocular discomfort (preferably for use in reducing discomfort during use of contact lenses), the composition containing (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil. Further, as one embodiment of the present invention, there is provided the use of (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil, for producing an aqueous ophthalmic composition for use in reducing ocular discomfort (preferably, for use in reducing discomfort during use of contact lenses). Further, as one embodiment of the present invention, there is provided a method of imparting an effect of reducing ocular discomfort (preferably, an effect of reducing discomfort during use of contact lenses) to an aqueous ophthalmic composition, comprising adding (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil to the aqueous ophthalmic composition. Further, as one embodiment of the present invention, there is provided a method of reducing discomfort during wearing contact lenses, comprising applying an aqueous ophthalmic composition containing (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil to contact lenses. Note that, in this method, the aqueous ophthalmic composition may be applied to the contact lenses during use of contact lenses or when wearing contact lenses.

Note that, in each of the embodiments, the kind and content of the components (A) and (B), the kind and content of other components, the use and dosage of the aqueous ophthalmic composition are as explained in [1. Aqueous Ophthalmic Composition]. Further, in the present embodiment, in view of more significantly exerting the effect of reducing ocular discomfort during wearing contact lenses, the aqueous ophthalmic composition is preferably used for contact lenses.

[3. Improvement in Eye Dryness and Eye Strain]

The aqueous ophthalmic composition according to the present embodiment produces an effect of reducing eye dryness (including evaporative dry eye) and eye strain.

Therefore, as one embodiment of the present invention, there is provided an agent for reducing eye dryness (evaporative dry eye or the like) and/or eye strain (preferably, an agent for reducing eye dryness during use of contact lenses (evaporative dry eye or the like) and/or eye strain) which is consisting of an aqueous ophthalmic composition containing (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil. As one embodiment of the present invention, there is provided an aqueous ophthalmic composition for use in improving eye dryness (evaporative dry eye or the like) and/or eye strain (preferably, to improve eye dryness during use of contact lenses (evaporative dry eye or the like) and/or eye strain), containing (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil. Further, as one embodiment of the present invention, there is provided the use of (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil for producing an aqueous ophthalmic composition for use in reducing eye dryness (evaporative dry eye or the like) and/or eye strain (preferably, to improve eye dryness during use of contact lenses (evaporative dry eye or the like) and/or eye strain). Further, as one embodiment of the present invention, there is provided a method of imparting an effect of reducing eye dryness (evaporative dry eye or the like) and/or eye strain (preferably, an effect of reducing eye dryness during use of contact lenses (evaporative dry eye or the like) and/or eye strain) to an aqueous ophthalmic composition, comprising adding (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil to the aqueous ophthalmic composition. Further, as one embodiment of the present invention, there is provided a method of reducing discomfort during use of contact lenses, comprising using an aqueous ophthalmic composition containing (A) vaseline and (B) at least one component selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil for contact lenses. Note that, in this method, the aqueous ophthalmic composition may be applied to the contact lenses during use of contact lenses or when wearing contact lenses.

Note that, in each of the embodiments, the kind and content of the components (A) and (B), the kind and content of other components, the use and dosage of the aqueous ophthalmic composition are as explained in [1. Aqueous Ophthalmic Composition]. Further, in the present embodiment, in view of more significantly exerting the effect of reducing ocular discomfort during wearing contact lenses, the aqueous ophthalmic composition is preferably used for contact lenses.

### EXAMPLES

Hereinafter, the present invention will be specifically explained based on Test Examples, however the present invention is not limited thereto.

Note that the light transmittance at a wavelength of 660 nm of the aqueous ophthalmic composition used in each of all the examples was 99.0% or more.

### Test Example 1: Dynamic Contact Angle (Angle of Advance) Evaluation 1

In accordance with the prescription described in Tables 1 to 3, aqueous ophthalmic compositions (eye drops) were prepared (the unit of each component amount in Tables 1 to 3 is w/v %). The contact angle meter DM-501 (manufactured by Kyowa Interface Science Co., Ltd.) was used to measure the dynamic contact angle of each of the ophthalmic solutions. The dynamic contact angle is a contact angle when the interface between solid and liquid moves.

The measurement was performed in accordance with the measurement procedure of dynamic contact angle (angle of advance) based on the expansion and contraction method using the contact angle meter. First, new hard contact lenses (Clear Blue (Group I, PMMA material), manufactured by Alpha Corporation) were sufficiently rinsed with purified water and placed on the stage of the contact angle meter after wiping off the water on the surface. A test solution (an eye drop) was placed in a dispenser of the contact angle meter. 1 μL of droplets of the test solution at room temperature was dropped onto hard contact lenses to form a hemispherical shape. Then, to the top of the hemispherical shape of the test solution, the head of the liquid discharge unit of the dispenser was attached. In the state, the test solution was continuously discharged at a discharge velocity of 6 μL/sec and the shapes of the droplets were photographed from the side surface 15 times per 0.1 sec. The photographed images were analyzed by the analysis software FAMAS of the contact angle meter, and the contact angle was calculated from each of the images. Here, among angles formed by the tangent line drawn from the contact point P of the hard contact lens surface, the test solution, and the air to the test solution and the tangent line drawn on the hard contact lens surface, the contact angle means an angle at the side including the test solution. The two contact points P were present at the right and left sides for each of the droplets and the average of the contact angles at the two points was calculated. After that, the contact angles (average of the two points) were arranged in the order of photographing the images, five consecutive contact angles were selected, and the standard deviation thereof was calculated. In other words, eleven standard deviations were calculated by a single measurement. The first contact angle when the standard deviation of the five consecutive contact angles firstly reached 2.5° or less was defined as the dynamic contact angle. Regarding all the test solutions, after the standard deviation of the five consecutive contact angles firstly reached 2.5° or less, no standard deviation larger than 2.5° was observed.

The measurement and calculation were repeatedly performed three times on each of the test solutions, and the standard deviation and average of the measured dynamic contact angles were calculated. Note that, in all the test solutions, the standard deviation of each of the three dynamic contact angles was less than 5.0°. Then, the improvement rate of the dynamic contact angle of each of the test examples relative to the corresponding comparative example was calculated by the formula below. The calculated results are shown in Tables 1 to 3.

Improvement rate (%)={1−(average of dynamic contact angles of test example/average of dynamic contact angles of corresponding comparative example)}×100

The corresponding comparative example means the aqueous ophthalmic composition containing neither the component (A) nor the component (B). Specifically, the corresponding comparative example is Comparative Example 1-1-1 for Comparative Examples 1-1-2 to 1-1-5 and Examples 1-1-1 to 1-1-3, the corresponding comparative example is Comparative Example 1-2-1 for Comparative Examples 1-2-2 and 1-2-3, and Example 1-2-1, the corresponding comparative example is Comparative Example 1-3-1 for Comparative Examples 1-3-2 and 1-3-3, and Example 1-3-1.

In Comparative Examples 1-3-1 to 1-3-3 and Example 1-3-1, 5 mL of each of the drug products of the test examples after preparation was filled in a 10 mL glass head space vial and allowed to stand at 50° C. for 6 days, i.e., heat-aged products were used.

TABLE 1

| | Com- | Com- | Com- | | Com- | | Com- | |
|---|---|---|---|---|---|---|---|---|
| | parative | parative | parative | | parative | | parative | |
| | Example | Example | Example | Example | Example | Example | Example | Example |
| | 1-1-1 | 1-1-2 | 1-1-3 | 1-1-1 | 1-1-4 | 1-1-2 | 1-1-5 | 1-1-3 |
| White vaseline | — | 0.001 | — | 0.001 | — | 0.001 | — | 0.001 |
| Polyvinylpyrrolidone K90 | — | — | 0.1 | 0.1 | — | — | — | — |
| Hydroxypropylmethylcellulose 2906 | — | — | — | — | 0.1 | 0.1 | — | — |
| Sodium hyaluronate | — | — | — | — | — | — | 0.002 | 0.002 |
| Boric acid | 1 | 1 | 1 | 1 | 1 | 1 | 1 | 1 |
| Borax | 0.08 | 0.08 | 0.08 | 0.08 | 0.08 | 0.08 | 0.08 | 0.08 |
| Polyoxyethylene hydrogenated castor | 0.2 | 0.2 | 0.2 | 0.2 | 0.2 | 0.2 | 0.2 | 0.2 |
| oil 60 | | | | | | | | |
| Hydrochloric acid | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Sodium hydroxide | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Purified water | Balance | Balance | Balance | Balance | Balance | Balance | Balance | Balance |
| pH | 7 | 7 | 7 | 7 | 7 | 7 | 7 | 7 |
| Improvement rate (%) | — | −2.7 | −9.8 | 2.9 | −1.1 | 6 | −9.2 | 0.5 |

TABLE 2

| | Comparative | Comparative | Comparative | |
|---|---|---|---|---|
| | Example | Example | Example | Example |
| | 1-2-1 | 1-2-2 | 1-2-3 | 1-2-1 |
| White vaseline | — | 0.001 | — | 0.001 |
| Polyvinylpyrrolidone K25 | — | — | 3 | 3 |
| Disodium hydrogenphosphate- | 1 | 1 | 1 | 1 |
| dodecahydrate | | | | |
| Sodium dihydrogen | 0.06 | 0.06 | 0.06 | 0.06 |
| phosphate-dihydrate | | | | |
| Polyoxyethylene hydrogenated | 0.2 | 0.2 | 0.2 | 0.2 |
| castor oil 60 | | | | |
| Hydrochloric acid | Appropriate | Appropriate | Appropriate | Appropriate |
| | amount | amount | amount | amount |
| Sodium hydroxide | Appropriate | Appropriate | Appropriate | Appropriate |
| | amount | amount | amount | amount |
| Purified water | Balance | Balance | Balance | Balance |
| pH | 7 | 7 | 7 | 7 |
| Improvement rate (%) | — | −11.2 | −5.3 | 2.5 |

TABLE 3

| | Comparative | Comparative | Comparative | |
|---|---|---|---|---|
| | Example | Example | Example | Example |
| | 1-3-1 | 1-3-2 | 1-3-3 | 1-3-1 |
| White vaseline | — | 0.001 | — | 0.001 |
| Sodium | — | — | 0.5 | 0.5 |
| chondroitin | | | | |
| sulfate | | | | |
| Boric acid | 1 | 1 | 1 | 1 |
| Borax | 0.08 | 0.08 | 0.08 | 0.08 |
| Polyoxyethylene | 0.2 | 0.2 | 0.2 | 0.2 |
| hydrogenated | | | | |
| castor oil 60 | | | | |
| Hydrochloric | Appropriate | Appropriate | Appropriate | Appropriate |
| acid | amount | amount | amount | amount |
| Sodium | Appropriate | Appropriate | Appropriate | Appropriate |
| hydroxide | amount | amount | amount | amount |
| Purified water | Balance | Balance | Balance | Balance |
| pH | 7 | 7 | 7 | 7 |
| Improvement | — | −10.4 | −2.9 | 8.2 |
| rate (%) | | | | |

It is found that there is a new problem as described below. In the case of singly using the component (A) and the component (B) (polyvinylpyrrolidone K90, hydroxypropylmethylcellulose, sodium hyaluronate, polyvinylpyrrolidone K25, sodium chondroitin sulfate), the improvement rate of the dynamic contact angle is decreased in both the case where the drug product of the test example is heat aged and the case where the drug product of the test example is not heat aged, compared to the eye drop containing neither the component (A) nor the component (B), i.e., a single use of the component (A) or the component (B) may give the effect of increasing discomfort to the eye drop.

Meanwhile, surprisingly, in the case of using the component (A) in combination with the component (B), it is confirmed that the improvement rate of the dynamic contact angle is improved, compared to the eye drop containing neither the component (A) nor the component (B), i.e., the fluidity of the liquid is improved, and the discomfort reducing effect is improved by applying eye drop.

### Test Example 2: Dynamic Contact Angle (Angle of Advance) Evaluation 2

The dynamic contact angle was evaluated in the same manner as in Test Example 1 except that the aqueous ophthalmic compositions (ophthalmic solutions) described in Tables 4 to 9 were used as the test solutions (the unit of each component amount in Tables 4 to 9 is w/v %).

The corresponding comparative example for Examples 2-1-1 to 2-1-9 is Comparative Example 2-1-1, the corresponding comparative example for Examples 2-2-1 to 2-2-4 is Comparative Example 2-2-1, the corresponding comparative example for Examples 2-3-1 to 2-3-4 is Comparative Example 2-3-1, the corresponding comparative example for Example 2-4-1 is Comparative Example 2-4-1, the corresponding comparative example for Examples 2-5-1 to 2-5-2 is Comparative Example 2-5-1, and the corresponding comparative example for Examples 2-6-1 to 2-6-2 is Comparative Example 2-6-1.

Further, in Comparative Example 2-5-1 and Examples 2-5-1 to 2-5-2, 5 mL of each of the drug products of the test examples after preparation was filled in a 10 mL glass head space vial and allowed to stand at 50° C. for 23 days, and the resulting products were used. In Comparative Example 2-6-1 and Examples 2-6-1 to 2-6-2, 5 mL of each of the drug products of the test examples after preparation was filled in a 10 mL glass head space vial and allowed to stand at 75° C. for 5 days, and the resulting products were used.

TABLE 4

| | Com- | | | | | | | | | |
|---|---|---|---|---|---|---|---|---|---|---|
| | parative | | | | | | | | | |
| | Example | Example | Example | Example | Example | Example | Example | Example | Example | Example |
| | 2-1-1 | 2-1-1 | 2-1-2 | 2-1-3 | 2-1-4 | 2-1-5 | 2-1-6 | 2-1-7 | 2-1-8 | 2-1-9 |
| White vaseline | — | 0.001 | 0.001 | 0.001 | 0.001 | 0.001 | 0.001 | 0.001 | 0.001 | 0.001 |
| Carboxyvinyl | — | 0.01 | — | — | — | — | — | — | — | — |
| polymer | | | | | | | | | | |
| Hydroxyethyl | — | — | 0.1 | — | — | — | — | — | — | — |
| cellulose | | | | | | | | | | |
| Alginic acid | — | — | — | 0.1 | — | — | — | — | — | — |
| Benzalkonium | — | — | — | — | 0.01 | — | — | — | — | — |
| chloride | | | | | | | | | | |
| Polyhexamethylene | — | — | — | — | — | 0.0001 | — | — | — | — |
| biguanide | | | | | | | | | | |
| Pyridoxine | — | — | — | — | — | — | 0.1 | — | — | — |
| hydrochloride | | | | | | | | | | |
| Neostigmine | — | — | — | — | — | — | — | 0.005 | — | — |
| methylsulfate | | | | | | | | | | |
| Naphazoline | — | — | — | — | — | — | — | — | 0.003 | — |
| hydrochloride | | | | | | | | | | |
| Poloxamer 407 | | | | | | | | | | 0.1 |
| Boric acid | 1 | 1 | 1 | 1 | 1 | 1 | 1 | 1 | 1 | 1 |
| Borax | 0.08 | 0.08 | 0.08 | 0.08 | 0.08 | 0.08 | 0.08 | 0.08 | 0.08 | 0.08 |
| Polyoxyethylene | 0.2 | 0.2 | 0.2 | 0.2 | 0.2 | 0.2 | 0.2 | 0.2 | 0.2 | 0.2 |
| hydrogenated | | | | | | | | | | |
| castor oil 60 | | | | | | | | | | |
| Hydrochloric acid | Appro- | Appro- | Appro- | Appro- | Appro- | Appro- | Appro- | Appro- | Appro- | Appro- |
| | priate | priate | priate | priate | priate | priate | priate | priate | priate | priate |
| | amount | amount | amount | amount | amount | amount | amount | amount | amount | amount |
| Sodium hydroxide | Appro- | Appro- | Appro- | Appro- | Appro- | Appro- | Appro- | Appro- | Appro- | Appro- |
| | priate | priate | priate | priate | priate | priate | priate | priate | priate | priate |
| | amount | amount | amount | amount | amount | amount | amount | amount | amount | amount |
| Purified water | Balance | Balance | Balance | Balance | Balance | Balance | Balance | Balance | Balance | Balance |
| pH | 7 | 7 | 7 | 7 | 7 | 7 | 7 | 7 | 7 | 7 |
| Improvement rate | — | 2 | 2.3 | 2.4 | 2.3 | 2.7 | 1.6 | 3 | 2.9 | 2.4 |
| (%) | | | | | | | | | | |

TABLE 5

| | Comparative | | | | |
|---|---|---|---|---|---|
| | Example | Example | Example | Example | Example |
| | 2-2-1 | 2-2-1 | 2-2-2 | 2-2-3 | 2-2-4 |
| White vaseline | — | 0.001 | 0.001 | 0.001 | 0.001 |
| Retinol palmitate | — | 50000 IU | — | — | — |
| Tocopherol acetate | — | — | 0.05 | — | — |
| Dibutylhydroxytoluene | — | — | — | 0.005 | — |
| Sesame oil | — | — | — | — | 0.05 |
| Boric acid | 1 | 1 | 1 | 1 | 1 |
| Borax | 0.08 | 0.08 | 0.08 | 0.08 | 0.08 |
| Polyoxyethylene | 0.4 | 0.4 | 0.4 | 0.4 | 0.4 |
| hydrogenated castor | | | | | |
| oil 60 | | | | | |
| Hydrochloric acid | Appropriate | Appropriate | Appropriate | Appropriate | Appropriate |
| | amount | amount | amount | amount | amount |
| Sodium hydroxide | Appropriate | Appropriate | Appropriate | Appropriate | Appropriate |
| | amount | amount | amount | amount | amount |
| Purified water | Balance | Balance | Balance | Balance | Balance |
| pH | 7 | 7 | 7 | 7 | 7 |
| Improvement rate (%) | — | 2.2 | 5 | 2.6 | 3.8 |

TABLE 6

| | Comparative | | | | |
|---|---|---|---|---|---|
| | Example | Example | Example | Example | Example |
| | 2-3-1 | 2-3-1 | 2-3-2 | 2-3-3 | 2-3-4 |
| White vaseline | — | 0.001 | 0.001 | 0.001 | 0.001 |
| Sodium chloride | — | 0.6 | — | — | — |
| Potassium chloride | — | — | 0.1 | — | — |
| Calcium chloride | — | — | — | 0.05 | — |
| Zinc chloride | — | — | — | — | 0.0025 |
| Polyoxyethylene | 0.2 | 0.2 | 0.2 | 0.2 | 0.2 |
| hydrogenated castor | | | | | |
| oil 60 | | | | | |
| Hydrochloric acid | Appropriate | Appropriate | Appropriate | Appropriate | Appropriate |
| | amount | amount | amount | amount | amount |
| Sodium hydroxide | Appropriate | Appropriate | Appropriate | Appropriate | Appropriate |
| | amount | amount | amount | amount | amount |
| Purified water | Balance | Balance | Balance | Balance | Balance |
| pH | 7 | 7 | 7 | 7 | 7 |
| Improvement rate (%) | — | 6.2 | 1.8 | 3.4 | 6.3 |

TABLE 7

| | Comparative | |
|---|---|---|
| | Example | Example |
| | 2-4-1 | 2-4-1 |
| White vaseline | — | 0.001 |
| Potassium L-aspartate | — | 1 |
| Sodium hydrogen phosphate | 1 | 1 |
| Sodium dihydrogen phosphate | 0.08 | 0.08 |
| Polyoxyethylene hydrogenated | 0.2 | 0.2 |
| castor oil 60 | | |
| Hydrochloric acid | Appropriate | Appropriate |
| | amount | amount |
| Sodium hydroxide | Appropriate | Appropriate |
| | amount | amount |
| Purified water | Balance | Balance |
| pH | 7 | 7 |
| Improvement rate (%) | — | 1.5 |

Note that, in Comparative Example 2-4-1 and Example 2-4-1, disodium hydrogenphosphate dodecahydrate was used as sodium hydrogen phosphate, and sodium dihydrogen phosphate dihydrate was used as sodium dihydrogen phosphate.

TABLE 8

| | Comparative | | |
|---|---|---|---|
| | Example | Example | Example |
| | 2-5-1 | 2-5-1 | 2-5-2 |
| White vaseline | — | 0.001 | 0.001 |
| Glucose | — | 0.05 | — |
| Taurine | — | — | 1 |
| Boric acid | 1 | 1 | 1 |
| Borax | 0.08 | 0.08 | 0.08 |
| Polyoxyethylene | 0.2 | 0.2 | 0.2 |
| hydrogenated castor | | | |
| oil 60 | | | |
| Hydrochloric acid | Appropriate | Appropriate | Appropriate |
| | amount | amount | amount |
| Sodium hydroxide | Appropriate | Appropriate | Appropriate |
| | amount | amount | amount |
| Purified water | Balance | Balance | Balance |
| pH | 7 | 7 | 7 |
| Improvement rate (%) | — | 3.9 | 5.6 |

TABLE 9

| | Comparative | | |
|---|---|---|---|
| | Example | Example | Example |
| | 2-6-1 | 2-6-1 | 2-6-2 |
| White vaseline | — | 0.001 | 0.001 |
| Propylene glycol | — | 0.5 | — |
| Polyethylene glycol 4000 | — | — | 0.5 |
| Boric acid | 1 | 1 | 1 |
| Borax | 0.08 | 0.08 | 0.08 |
| Polyoxyethylene | 0.2 | 0.2 | 0.2 |
| hydrogenated castor | | | |
| oil 60 | | | |
| Hydrochloric acid | Appropriate | Appropriate | Appropriate |
| | amount | amount | amount |
| Sodium hydroxide | Appropriate | Appropriate | Appropriate |
| | amount | amount | amount |
| Purified water | Balance | Balance | Balance |
| pH | 7 | 7 | 7 |
| Improvement rate (%) | — | 23 | 2 |

In the case of using carboxyvinyl polymer, hydroxyethyl cellulose, alginic acid, benzalkonium chloride, polyhexamethylene biguanide, pyridoxine hydrochloride, neostigmine methylsulfate, naphazoline hydrochloride, Poloxamer 407, retinol palmitate, tocopherol acetate, dibutylhydroxytoluene, sesame oil, sodium chloride, potassium chloride, calcium chloride, zinc chloride, potassium L-aspartate, glucose, taurine, propylene glycol or polyethylene glycol 4000 as the component (B), similarly to Test Example 1, in the case of singly using the component (A) and the component (B) singly, despite the fact that the improvement rate of the dynamic contact angle is improved, compared to the eye drop containing neither the component (A) nor the component (B), in the case of using the component (A) in combination with the component (B), it is confirmed that the improvement rate of the dynamic contact angle is improved, compared to the eye drop containing neither the component (A) nor the component (B), i.e., the fluidity of the liquid is improved, and the discomfort reducing effect is improved by applying eye drop.

Note that, in the cases where tests were performed in the same manner as in Test Examples 1 and 2 except that cultured corneal cells were used in place of the hard contact lens, the same results were obtained.

### Test Example 3: Eye Drop Test in Humans

In accordance with the prescription described in Tables 10 to 11, aqueous ophthalmic compositions (eye drops) were prepared and 13 mL of each of the compositions was filled in a 14.2-mL container for eye drops made of polyethylene terephthalate (the unit of each component amount in Tables 10 and 11 is w/v %). After being filled, a polyethylene nozzle was attached to the container for eye drops. One drop of each of the eye drops was dropped onto the right and left eyes of six subjects and the feelings after applying eye drop described in Tables 10 and 11 were evaluated by the visual analog scale (VAS). Among six subjects, three subjects wore soft contact lenses (SCL), (two subjects wore Acuvue Oasys (Group I, senofilcon A) manufactured by Johnson & Johnson, and one subject wore Air Optix Aqua (Group 1, lotrafilcon B) manufactured by Alcon Japan), and three subjects wore no glasses.

The evaluation based on VAS was performed in the following manner. On a sheet with a 100 mm line to examine subjective symptoms, the case of having no feeling regarding the sensory items described in Tables 10 and 11 was 0 mm and the case of having strong feelings was 100 mm, and then a mark was placed on the level of each of the items in which each subject felt. This length (mm) was defined as the VAS value. In other words, the higher the VAS value, the higher the sensory evaluation scores.

TABLE 10

| | | | Comparative | Example |
|---|---|---|---|---|
| | | | Example 3-1 | 3-1 |
| White vaseline | | | — | 0.001 |
| Hydroxypropylmethylcellulose 2906 | | | 0.1 | 0.1 |
| Sodium chloride | | | 0.4 | 0.4 |
| Boric acid | | | 1 | 1 |
| Borax | | | 0.08 | 0.08 |
| Polyoxyethylene hydrogenated castor | | | 0.2 | 0.2 |
| oil 60 | | | | |
| Hydrochloric acid | | | Appropriate | Appropriate |
| | | | amount | amount |
| Sodium hydroxide | | | Appropriate | Appropriate |
| | | | amount | amount |
| Purified water | | | Balance | Balance |
| pH | | | 7.2 | 7.2 |
| VAS value | Immediately | Feeling of | — | 59.5 |
| (difference | after | reduced | | |
| from | applying | discomfort | | |
| Comparative | eye drops | Feeling of | — | 76.7 |
| Example | | smooth | | |
| 3-1) | | blinking | | |
| | | Feeling of | — | 77.5 |
| | | smoothness | | |
| | | when | | |
| | | blinking | | |
| | | Feeling of | — | 65.1 |
| | | clear vision | | |
| | 15 minutes | Feeling of | — | 55.1 |
| | after | reduced | | |
| | applying | discomfort | | |
| | eye drops | Feeling of | — | 53 |
| | | smooth | | |
| | | blinking | | |
| | | Feeling of | — | 56.6 |
| | | smoothness | | |
| | | when | | |
| | | blinking | | |
| | | Feeling of | — | 52.3 |
| | | clear vision | | |
| | | Feeling of | — | −42.2 |
| | | drying | | |
| | | Feeling of | — | −43.9 |
| | | eyestrain | | |

TABLE 11

| | | | Comparative | Example |
|---|---|---|---|---|
| | | | Example 3-1 | 3-1 |
| White vaseline | | | 0.001 | 0.001 |
| Tetrahydrozoline hydrochloride | | | — | 0.05 |
| Neostigmine methylsulfate | | | — | 0.005 |
| Epsilon-aminocaproic acid | | | — | 1 |
| Dipotassium glycyrrhizinate | | | — | 0.2 |
| Potassium L-aspartate | | | — | 1 |
| Taurine | | | 1 | — |
| Sodium chondroitin sulfate | | | 0.5 | — |
| Pyridoxine hydrochloride | | | — | 0.1 |
| Benzalkonium chloride | | | — | 0.01 |
| Dextran 70 | | | 0.1 | — |
| Propylene glycol | | | — | 0.2 |
| Sodium chloride | | | 0.3 | — |
| Potassium chloride | | | 0.1 | — |
| Calcium chloride | | | 0.015 | — |
| Boric acid | | | 0.5 | 0.5 |
| Borax | | | 0.07 | 0.03 |
| Citric acid | | | — | 0.05 |
| Edetate sodium | | | 0.1 | 0.05 |
| 1-menthol | | | — | 0.02 |
| d-camphor | | | — | 0.01 |
| d-borneol | | | — | 0.005 |
| Polyoxyethylene hydrogenated castor | | | 0.2 | 0.2 |
| oil 60 | | | | |
| Polysorbate 80 | | | — | 0.2 |
| Chlorobutanol | | | — | 0.2 |
| Purified water | | | Balance | Balance |
| pH | | | 6.9 | 5.4 |
| Osmotic pressure ratio | | | 1 | 1.2 |
| VAS | Immediately | Feeling of reduced | 91.2 | 91 |
| value | after | discomfort | | |
| | applying | Feeling of smooth | 88.4 | 90.5 |
| | eye drops | blinking | | |
| | | Feeling of smoothness | 89 | 87.1 |
| | | when blinking | | |
| | | Feeling of clear vision | 81.5 | 91.6 |
| | 15 minutes | Feeling of reduced | 90.3 | 89.5 |
| | after | discomfort | | |
| | applying | Feeling of smooth | 83.9 | 82.7 |
| | eye drops | blinking | | |
| | | Feeling of smoothness | 75.6 | 79.1 |
| | | when blinking | | |
| | | Feeling of clear vision | 73.9 | 86.6 |
| | | Feeling of drying | 9.8 | 13 |
| | | Feeling of eyestrain | 10.8 | 7.5 |

The results of Tables 10 and 11 show that, in the case of using the component (A) in combination with the component (B), indicators of ocular discomfort, smooth blinking, and smoothness when blinking, and clear vision (immediately after applying eye drops) and indicators of ocular discomfort, smooth blinking, smoothness when blinking, clear vision, feeling of drying, and eye strain (15 minutes after applying eye drops) tend to be significantly improved, compared to the ophthalmic solution containing only the component (B).

Note that the tendency of significantly exerting the effect was observed in only subjects wearing SCL. Specifically, in only subjects wearing SCL, in Example 3-1, the VAS values (difference from Comparative Example 3-1) were 86.4 (feeling of reduced discomfort) and 70.5 (feeling of reduced discomfort 15 minutes after applying eye drops). In Example 3-2, the VAS values were 95.9 (feeling of reduced discomfort) and 95.9 (feeling of reduced discomfort 15 minutes after applying eye drops). In Example 3-3, the VAS values were 98.4 (feeling of reduced discomfort) and 95.1 (feeling of reduced discomfort 15 minutes after applying eye drops).

### Test Example 4: Dynamic Contact Angle (Angle of Advance) Evaluation of 3

In accordance with the prescription described in Tables 12 to 13, aqueous ophthalmic compositions (eye drops) were prepared (the unit of each component amount in Tables 12 to 13 is w/v %). The contact angle meter DM-501 (manufactured by Kyowa Interface Science Co., Ltd.) was used to measure the dynamic contact angle of each of the eye drops to the cells. The dynamic contact angle is a contact angle when the interface between solid and liquid moves.

The used cells were cultured corneal cells. Human corneal epithelial cell lines HCE-T (No. RCB2280, Riken BioResource Center) were seeded in a 2-well culture slide chamber (LAB-TEKIIRS Glass, manufactured by Thermo Fisher Scientific Inc.) at 1.0×10⁵ cells/well and cultured at 37° C. in 5% CO₂ at a humidity of 90% for 72 hours. The used culture solution was a DMEM/F-12 medium (GIBCO) containing 5% fetal calf serum (FCS). After culturing, a procedure of pouring purified water (2 mL/well) into each well to allow the cells to be immersed and aspirating and removing the purified water immediately was repeated twice, followed by washing. After washing, the 2-well culture slide chamber was removed. After being allowed to stand at room temperature for 30 minutes, the contact angle was evaluated.

The measurement was performed in accordance with the measurement procedure of the drop method using the contact angle meter. First, the culture slide in which the cells had been cultured was placed on the stage of the contact angle meter. A test solution (an eye drop) was placed in a dispenser of the contact angle meter. 1 μL of droplets of the test solution at room temperature was dropped onto the cells to form a hemispherical shape. The droplets on the cells spread with the passage of time, thereby changing the shapes and contact angles of the droplets. Immediately after being dropped, the shapes of the droplets were photographed from the side surface 15 times per 0.1 sec. The photographed images were analyzed by the analysis software FAMAS of the contact angle meter, and the contact angle was calculated from each of the images. Here, among angles formed by the tangent line drawn from the contact point P of the cell surface, the test solution, and air to the test solution and the tangent line drawn on the cell surface, the contact angle means an angle at the side including the test solution. The two contact points P were present at the right and left sides for each of the droplets and the average of the contact angles at the two points was calculated. After that, the contact angles (average of the two points) were arranged in the order of photographing the images, five consecutive contact angles were selected, and the standard deviation thereof was calculated. In other words, eleven standard deviations were calculated by a single measurement. The first contact angle when the standard deviation of the five consecutive contact angles firstly reached 1.0° or less was defined as the dynamic contact angle. Regarding all the test solutions, after the standard deviation of the five consecutive contact angles firstly reached 1.0° or less, no standard deviation larger than 1.0° was observed.

The measurement and calculation were repeatedly performed three times on each of the test solutions, and the standard deviation and average of the measured dynamic contact angles were calculated. Note that, in all the test solutions, the standard deviation of each of the three dynamic contact angles was less than 1.5°. Then, the improvement rate of the dynamic contact angle of each of the test examples relative to the corresponding comparative example was calculated by the formula below. The calculated results are shown in Tables 12 and 13.

Improvement rate (%)={1−(average of dynamic contact angles of test example/average of dynamic contact angles of corresponding comparative example)}×100

The corresponding comparative example means the aqueous ophthalmic composition containing neither the component (A) nor the component (B). Specifically, the corresponding comparative example for Comparative Example 4-1-2 and Examples 4-1-1 to 4-1-7 is Comparative Example 4-1-1, and the corresponding comparative example for Example 4-2-1 is Comparative Example 4-2-1.

TABLE 12

| | Com- | Com- | | | | | | | |
|---|---|---|---|---|---|---|---|---|---|
| | parative | parative | | | | | | | |
| | Example | Example | Example | Example | Example | Example | Example | Example | Example |
| | 4-1-1 | 4-1-2 | 4-1-1 | 4-1-2 | 4-1-3 | 4-1-4 | 4-1-5 | 4-1-6 | 4-1-7 |
| White vaseline | — | 0.001 | 0.001 | 0.001 | 0.001 | 0.001 | 0.001 | 0.001 | 0.001 |
| Hydroxypropylmethylcellulose 2906 | — | — | 0.1 | — | — | — | — | — | — |
| Polyvinylpyrrolidone K25 | — | — | — | 1 | — | — | — | — | — |
| Sodium chondroitin sulfate | — | — | — | — | 0.5 | — | — | — | — |
| Propylene glycol | — | — | — | — | — | 0.5 | — | — | — |
| Polyhexamethylene biguanide | — | — | — | — | — | — | 0.00008 | — | — |
| Benzalkonium chloride | — | — | — | — | — | — | — | 0.01 | — |
| Poloxamer 407 | — | — | — | — | — | — | — | — | 0.1 |
| Polyoxyethylene hydrogenated | 0.2 | 0.2 | 0.2 | 0.2 | 0.2 | 0.2 | 0.2 | 0.2 | 0.2 |
| castor oil 60 | | | | | | | | | |
| Hydrochloric acid | Appro- | Appro- | Appro- | Appro- | Appro- | Appro- | Appro- | Appro- | Appro- |
| | priate | priate | priate | priate | priate | priate | priate | priate | priate |
| | amount | amount | amount | amount | amount | amount | amount | amount | amount |
| Sodium hydroxide | Appro- | Appro- | Appro- | Appro- | Appro- | Appro- | Appro- | Appro- | Appro- |
| | priate | priate | priate | priate | priate | priate | priate | priate | priate |
| | amount | amount | amount | amount | amount | amount | amount | amount | amount |
| Purified water | Balance | Balance | Balance | Balance | Balance | Balance | Balance | Balance | Balance |
| pH | 7 | 7 | 7 | 7 | 7 | 7 | 7 | 7 | 7 |
| Improvement rate (%) | — | −2.5 | 9.8 | 3.5 | 3.7 | 9.3 | 8.6 | 3.1 | 9.9 |

TABLE 13

| | Comparative | |
|---|---|---|
| | Example | Example |
| | 4-2-1 | 4-2-1 |
| White vaseline | — | 0.001 |
| Neostigmine methylsulfate | — | 0.005 |
| Polyoxyethylene hydrogenated | 0.2 | 0.2 |
| castor oil 60 | | |
| Hydrochloric acid | Appropriate | Appropriate |
| | amount | amount |
| Sodium hydroxide | Appropriate | Appropriate |
| | amount | amount |
| Purified water | Balance | Balance |
| pH | 5 | 5 |
| Improvement rate (%) | — | 3.5 |

In the case of singly using the component (A) and the component (B) (hydroxypropylmethylcellulose 2906, polyvinylpyrrolidone K25, sodium chondroitin sulfate, propylene glycol, polyhexamethylene biguanide, benzalkonium chloride, Poloxamer 407), the improvement rate of the dynamic contact angle to the corneal cells is decreased, compared to the eye drops containing neither the component (A) nor the component (B). Meanwhile, in the case of using the component (A) in combination with the component (B), it is confirmed that the improvement rate of the dynamic contact angle to the corneal cells is improved, compared to the eye drops containing neither the component (A) nor the component (B), i.e., the fluidity of the liquid is improved, and the discomfort reducing effect is improved by applying eye drops (the naked eyes).

### Test Example 5: Dynamic Contact Angle (Angle of Advance) Evaluation 4

The dynamic contact angle was evaluated in the same manner as in Test Example 1 except that the aqueous ophthalmic compositions (eye drops) described in Tables 14 to 18 were used as the test solutions (the unit of each component amount in Tables 14 to 18 is w/v %). Note that, in all the test solutions, the standard deviation of each of the three dynamic contact angles was less than 2.0°.

The corresponding comparative example for Comparative Example 5-1-2 and Examples 5-1-1 to 5-1-5 is Comparative Example 5-1-1, the corresponding comparative example for Comparative Example 5-2-2 and Examples 5-2-1 to 5-2-3 is Comparative Example 5-2-1, the corresponding comparative example for Comparative Example 5-3-2 and Example 5-3-1 is Comparative Example 5-3-1, the corresponding comparative example for Comparative Example 5-4-2 and Examples 5-4-1 to 5-4-3 is Comparative Example 5-4-1, the corresponding comparative example for Comparative Example 5-5-1 and Example 5-5-2 is Comparative Example 5-5-1, the corresponding comparative example for Examples 5-6-1 and 5-6-2 is Comparative Example 5-6-1, the corresponding comparative example for Examples 5-7-1 and 5-7-2 is Comparative Example 5-7-1, the corresponding comparative example for Examples 5-8-1 and 5-8-2 is Comparative Example 5-8-1, the corresponding comparative example for Example 5-9-1 is Comparative Example 5-9-1, and the corresponding comparative example for Example 5-10-1 is Comparative Example 5-10-1.

TABLE 14

| | Com- | Com- | | | | | |
|---|---|---|---|---|---|---|---|
| | parative | parative | | | | | |
| | Example | Example | Example | Example | Example | Example | Example |
| | 5-1-1 | 5-1-2 | 5-1-1 | 5-1-2 | 5-1-3 | 5-1-4 | 5-1-5 |
| White vaseline | — | 0.001 | 0.001 | 0.001 | 0.001 | 0.001 | 0.001 |
| Chlorhexidine glyconate | — | — | 0.01 | — | — | — | — |
| D-mannitol | — | — | — | 3.5 | — | — | — |
| Carboxymethylcellulose sodium | — | — | — | — | 0.1 | — | — |
| Hydroxypropylmethylcellulose | — | — | — | — | — | 1.5 | — |
| 2910 | | | | | | | |
| Cyanocobalamin | — | — | — | — | — | — | 0.02 |
| Boric acid | 0.7 | 0.7 | 0.7 | 0.7 | 0.7 | 0.7 | 0.7 |
| Borax | 0.04 | 0.04 | 0.04 | 0.04 | 0.04 | 0.04 | 0.04 |
| Polysorbate 80 | 0.3 | 0.3 | 0.3 | 0.3 | 0.3 | 0.3 | 0.3 |
| Polyoxyl 40 stearate | — | — | — | — | — | 0.05 | — |
| Hydrochloric acid | Appro- | Appro- | Appro- | Appro- | Appro- | Appro- | Appro- |
| | priate | priate | priate | priate | priate | priate | priate |
| | amount | amount | amount | amount | amount | amount | amount |
| Sodium hydroxide | Appro- | Appro- | Appro- | Appro- | Appro- | Appro- | Appro- |
| | priate | priate | priate | priate | priate | priate | priate |
| | amount | amount | amount | amount | amount | amount | amount |
| Purified water | Balance | Balance | Balance | Balance | Balance | Balance | Balance |
| pH | 7 | 7 | 7 | 7 | 7 | 7 | 7 |
| Improvement rate (%) | — | −10.7 | 4.4 | 4.2 | 10.1 | 8.9 | 17.1 |

Carboxymethylcellulose sodium: AG gum M (DKS Co. LTD.)
Hydroxypropylmethylcellulose 2910: Metolose TC-5E (Shin-Etsu Chemical Co., Ltd.)

TABLE 15

| | Com- | Com- | | | | Com- | Com- | |
|---|---|---|---|---|---|---|---|---|
| | parative | parative | | | | parative | parative | |
| | Example | Example | Example | Example | Example | Example | Example | Example |
| | 5-2-1 | 5-2-2 | 5-2-1 | 5-2-2 | 5-2-3 | 5-3-1 | 5-3-2 | 5-3-1 |
| White vaseline | — | 0.001 | 0.001 | 0.001 | 0.001 | — | 0.001 | 0.001 |
| Sulfamethoxazole | — | — | 4 | — | — | — | — | — |
| sodium | | | | | | | | |
| Gellant gum | — | — | — | 0.08 | — | — | — | — |
| Polyethylene | — | — | — | — | 0.4 | — | — | — |
| glycol 400 | | | | | | | | |
| Monoethanolamine | — | — | — | — | — | — | — | 0.5 |
| Boric acid | 0.4 | 0.4 | 0.4 | 0.4 | 0.4 | 0.8 | 0.8 | 0.8 |
| Borax | 0.05 | 0.05 | 0.05 | 0.05 | 0.05 | 0.04 | 0.04 | 0.04 |
| Polysorbate 80 | 0.2 | 0.2 | 0.2 | 0.2 | 0.2 | 0.5 | 0.5 | 0.5 |
| Edetate sodium | — | — | — | 0.08 | — | — | — | — |
| Hydrochloric acid | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Sodium hydroxide | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Purified water | Balance | Balance | Balance | Balance | Balance | Balance | Balance | Balance |
| pH | 8 | 8 | 8 | 8 | 8 | 6.2 | 6.2 | 6.2 |
| Improvement rate | — | −6.5 | 5.9 | 2.8 | 8.4 | — | −6.0 | 6.2 |
| (%) | | | | | | | | |

TABLE 16

| | Com- | Com- | | | | Com- | | |
|---|---|---|---|---|---|---|---|---|
| | parative | parative | | | | parative | | |
| | Example | Example | Example | Example | Example | Example | Example | Example |
| | 5-4-1 | 5-4-2 | 5-4-1 | 5-4-2 | 5-4-3 | 5-5-1 | 5-5-1 | 5-5-2 |
| White vaseline | — | 0.001 | 0.001 | 0.001 | 0.001 | — | 0.001 | 0.001 |
| Dextran 70 | — | — | 0.05 | — | — | — | — | — |
| Taurine | — | — | — | 0.1 | — | — | — | — |
| Benzalkonium | — | — | — | — | 0.0001 | — | — | — |
| chloride | | | | | | | | |
| Poloxamer 188 | — | — | — | — | — | — | 5 | — |
| Trometamol | — | — | — | — | — | — | — | 1 |
| Boric acid | 0.5 | 0.5 | 0.5 | 0.5 | 0.5 | — | — | — |
| Borax | 0.01 | 0.01 | 0.01 | 0.01 | 0.01 | — | — | — |
| Polysorbate 80 | 0.3 | 0.3 | 0.3 | 0.3 | 0.3 | 1 | 1 | 1 |
| Hydrochloric | Appro- | Appro- | Appro- | Appro- | Appro- | Appro- | Appro- | Appro- |
| acid | priate | priate | priate | priate | priate | priate | priate | priate |
| | amount | amount | amount | amount | amount | amount | amount | amount |
| Sodium | Appro- | Appro- | Appro- | Appro- | Appro- | Appro- | Appro- | Appro- |
| hydroxide | priate | priate | priate | priate | priate | priate | priate | priate |
| | amount | amount | amount | amount | amount | amount | amount | amount |
| Purified water | Balance | Balance | Balance | Balance | Balance | Balance | Balance | Balance |
| pH | 5.2 | 5.2 | 5.2 | 5.2 | 5.2 | 7 | 7 | 7 |
| Improvement | — | −2.5 | 4.3 | 9.2 | 10.1 | — | 8.1 | 9.1 |
| rate (%) | | | | | | | | |

TABLE 17

| | Com- | | | Com- | | | Com- | | |
|---|---|---|---|---|---|---|---|---|---|
| | parative | | | parative | | | parative | | |
| | Example | Example | Example | Example | Example | Example | Example | Example | Example |
| | 5-6-1 | 5-6-1 | 5-6-2 | 5-7-1 | 5-7-1 | 5-7-2 | 5-8-1 | 5-8-1 | 5-8-2 |
| White vaseline | — | 0.05 | 0.05 | — | 0.1 | 0.1 | — | 0.25 | 0.25 |
| Sesame oil | — | 0.25 | 0.25 | — | 0.5 | 0.5 | — | 1.25 | 1.25 |
| Sodium chondroitin sulfate | — | — | 0.1 | — | — | — | — | — | — |
| Hydroxypropylmethylcellulose | — | — | — | — | — | 0.05 | — | — | — |
| 2906 | | | | | | | | | |
| Naphazoline hydrochloride | — | — | — | — | — | — | — | — | 0.03 |
| Boric acid | 0.7 | 0.7 | 0.7 | 0.4 | 0.4 | 0.4 | 2 | 2 | 2 |
| Borax | 0.04 | 0.04 | 0.04 | 0.05 | 0.05 | 0.05 | 0.05 | 0.05 | 0.05 |
| Polysorbate 80 | 1 | 1 | 1 | 2 | 2 | 2 | 5 | 5 | 5 |
| Polyoxyethylene hydrogenated | — | — | — | 0.5 | 0.5 | 0.5 | — | — | — |
| castor oil 60 | | | | | | | | | |
| Polyoxyethylene castor oil 10 | 1 | 1 | 1 | 2 | 2 | 2 | 5 | 5 | 5 |
| Hydrochloric acid | Appro- | Appro- | Appro- | Appro- | Appro- | Appro- | Appro- | Appro- | Appro- |
| | priate | priate | priate | priate | priate | priate | priate | priate | priate |
| | amount | amount | amount | amount | amount | amount | amount | amount | amount |
| Sodium hydroxide | Appro- | Appro- | Appro- | Appro- | Appro- | Appro- | Appro- | Appro- | Appro- |
| | priate | priate | priate | priate | priate | priate | priate | priate | priate |
| | amount | amount | amount | amount | amount | amount | amount | amount | amount |
| Purified water | Balance | Balance | Balance | Balance | Balance | Balance | Balance | Balance | Balance |
| pH | 7 | 7 | 7 | 8 | 8 | 8 | 5.2 | 5.2 | 5.2 |
| Improvement rate (%) | — | 2.3 | 7.7 | — | 5.6 | 9.0 | — | 6.5 | 10.4 |

TABLE 18

| | Comparative | | Comparative | |
|---|---|---|---|---|
| | Example | Example | Example | Example |
| | 5-9-1 | 5-9-1 | 5-10-1 | 5-10-1 |
| White vaseline | — | 0.001 | — | 0.001 |
| Glycerin | — | 0.5 | — | — |
| Magnesium | — | — | — | 0.015 |
| sulfate | | | | |
| Boric acid | 1 | 1 | — | — |
| Borax | 0.08 | 0.08 | — | — |
| Polyoxyethylene | 0.2 | 0.2 | 0.2 | 0.2 |
| hydrogenated | | | | |
| castor oil 60 | | | | |
| Hydrochloric | Appropriate | Appropriate | Appropriate | Appropriate |
| acid | amount | amount | amount | amount |
| Sodium | Appropriate | Appropriate | Appropriate | Appropriate |
| hydroxide | amount | amount | amount | amount |
| Purified water | Balance | Balance | Balance | Balance |
| pH | 7 | 7 | 7 | 7 |
| Improvement | — | 8.2 | — | 3.9 |
| rate (%) | | | | |

In the case of using chlorhexidine glyconate, D-mannitol, carboxymethylcellulose sodium, hydroxypropylmethylcellulose 2910, cyanocobalamin, sulfamethoxazole sodium, gellant gum, polyethylene glycol 400, monoethanolamine, dextran 70, taurine, benzalkonium chloride, Poloxamer-188, trometamol, glycerin or magnesium sulfate as the component (B), similarly to Test Example 1, in the case of singly using the component (A) and the component (B) singly, the improvement rate of the dynamic contact angle is decreased, compared to the eye drops containing neither the component (A) nor the component (B). While, in the case of using the component (A) in combination with the component (B), it is confirmed that the improvement rate of the dynamic contact angle is improved, compared to the eye drops containing neither the component (A) nor the component (B), i.e., the fluidity of the liquid is improved, and the discomfort reducing effect is improved by applying eye drops.

### Test Example 6: Eye Drop Test in Humans 2

The evaluation based on VAS was performed in the same manner as in Test Example 3 except that the aqueous ophthalmic compositions (eye drops) described in Tables 19 and 20 were used as the test solutions (the unit of each component amount in Tables 19 and 20 is w/v %). Note that the corresponding comparative example is the prescription in which the component (A) is removed from the prescription of each example and the pH is adjusted by hydrochloric acid and sodium hydroxide (the balance is purified water).

TABLE 19

| | | | Example | Example |
|---|---|---|---|---|
| | | | 6-1 | 6-2 |
| White vaseline | | | 0.005 | 0.005 |
| Polyvinylpyrrolidone k25 | | | 2 | — |
| Propylene glycol | | | — | 1 |
| Boric acid | | | 0.8 | 0.6 |
| Borax | | | 0.01 | 0.01 |
| Polysorbate 80 | | | 1 | 1 |
| Sodium chloride | | | 0.5 | — |
| Hydrochloric acid | | | Appropriate | Appropriate |
| | | | amount | amount |
| Sodium hydroxide | | | Appropriate | Appropriate |
| | | | amount | amount |
| Purified water | | | Balance | Balance |
| pH | | | 8 | 6.1 |
| VAS value | Immediately | Feeling of | 58.6 | 56.9 |
| (difference | after | reduced | | |
| from | applying | discomfort | | |
| corresponding | eye drops | Feeling of | 55.8 | 50.8 |
| comparative | | smooth | | |
| example) | | blinking | | |
| | | Feeling of | 51.1 | 42.8 |
| | | smoothness | | |
| | | when blinking | | |
| | | Feeling of | 51.7 | 41.3 |
| | | clear vision | | |
| | 15 minutes | Feeling of | 57.4 | 58.2 |
| | after | reduced | | |
| | applying | discomfort | | |
| | eye drops | Feeling of | 51.5 | 43.6 |
| | | smooth | | |
| | | blinking | | |
| | | Feeling of | 48.7 | 48.0 |
| | | smoothness | | |
| | | when blinking | | |
| | | Feeling of | 48.1 | 43.9 |
| | | clear vision | | |
| | | Feeling of | −42.6 | −38.0 |
| | | drying | | |
| | | Feeling of | −39.2 | −32.0 |
| | | eyestrain | | |

TABLE 20

| | | | Example | Example |
|---|---|---|---|---|
| | | | 6-3 | 6-4 |
| White vaseline | | | 0.005 | 0.005 |
| Cyanocobalamin | | | 0.02 | — |
| Neostigmine methylsulfate | | | — | 0.005 |
| Boric acid | | | — | 0.8 |
| Borax | | | — | 0.01 |
| Disodium hydrogenphosphate | | | 1.3 | — |
| dodecahydrate | | | | |
| Sodium dihydrogen phosphate dihydrate | | | 0.1 | — |
| Polysorbate 80 | | | 1 | 1 |
| Sodium chloride | | | 0.6 | 0.2 |
| Hydrochloric acid | | | Appropriate | Appropriate |
| | | | amount | amount |
| Sodium hydroxide | | | Appropriate | Appropriate |
| | | | amount | amount |
| Purified water | | | Balance | Balance |
| pH | | | 6.2 | 5.2 |
| VAS value | Immediately | Feeling of | 45.9 | 39.2 |
| (difference | after | reduced | | |
| from | applying | discomfort | | |
| corresponding | eye drops | Feeling of | 33.9 | 25.4 |
| comparative | | smooth | | |
| example) | | blinking | | |
| | 15 minutes | Feeling of | 45.4 | 40.2 |
| | after | reduced | | |
| | applying | discomfort | | |
| | eye drops | Feeling of | 39.1 | 35.4 |
| | | smooth | | |
| | | blinking | | |
| | | Feeling of | −37.9 | −35.1 |
| | | drying | | |
| | | Feeling of | −41.7 | −42.8 |
| | | eyestrain | | |

The results of Tables 19 and 20 show that, in the case of using the component (A) in combination with the component (B), indicators of ocular discomfort, smooth blinking, and smoothness when blinking, and clear vision (immediately after applying eye drops) and indicators of ocular discomfort, smooth blinking, smoothness when blinking, clear vision, feeling of drying, and eye strain (15 minutes after applying eye drops) tend to be significantly improved, compared to the eye drops containing only the component (B).

### Preparation Examples

Eye drops are prepared according to the prescription described in Tables 21 to 23. All the units in each of the tables are (w/v %). The eye drops prescribed in Preparation Examples 1 to 24 were filled in polyethylene terephthalate containers, and then polyethylene nozzles were attached thereto.

TABLE 21

| | Prepa- | Prepa- | Prepa- | Prepa- | Prepa- | Prepa- | Prepa- | Prepa- |
|---|---|---|---|---|---|---|---|---|
| | ration | ration | ration | ration | ration | ration | ration | ration |
| | Example | Example | Example | Example | Example | Example | Example | Example |
| | 1 | 2 | 3 | 4 | 5 | 6 | 7 | 8 |
| White vaseline | 0.1 | 1 | 0.005 | 0.001 | 0.001 | 0.001 | 0.01 | 0.01 |
| Tetrahydrozoline hydrochloride | — | — | — | 0.05 | 0.03 | — | 0.05 | — |
| Naphazoline hydrochloride | — | 0.003 | — | — | — | — | — | — |
| Neostigmine methylsulfate | — | — | — | — | — | 0.005 | 0.005 | — |
| Disodium cromoglycate | — | — | — | — | — | — | — | 1 |
| Chlorpheniramine maleate | — | — | — | 0.03 | 0.03 | 0.01 | 0.03 | 0.015 |
| Pranoprofen | — | — | — | — | — | — | — | 0.05 |
| Epsilon aminocaproic acid | — | 1 | 0.9 | 0.9 | 1.5 | 0.7 | 0.8 | — |
| Allantoin | — | — | — | — | — | — | 0.3 | — |
| Azulene sulfonate sodium | 0.004 | — | — | — | 0.02 | — | — | — |
| Glicyrrhizic acid 2K | 0.05 | — | — | — | 0.2 | 0.1 | 0.1 | — |
| Zinc sulfate | — | — | — | 0.1 | — | — | 0.05 | — |
| Flavin adenin dinucleotide sodium | — | 0.005 | — | — | — | — | — | — |
| Cyanocobalamin | — | — | — | 0.02 | — | — | 0.01 | — |
| Pyridoxine hydrochloride | — | — | — | 0.05 | 0.1 | 0.1 | 0.1 | — |
| d-α-tocopheryl acetate | — | — | — | — | — | — | 0.05 | — |
| Panthenol | — | — | — | — | 0.1 | 0.1 | — | — |
| Potassium aspartate | — | — | — | 1 | — | — | 1 | — |
| Magnesium aspartate | 0.5 | 0.01 | — | — | — | — | — | — |
| Aminoethylsulfonic acid | — | — | — | 1 | 0.1 | 1 | 0.5 | — |
| Sodium chondroitin sulfate | — | 3 | — | 0.5 | — | 0.5 | 0.1 | 0.5 |
| Polyvinyl alcohol | — | 1 | — | — | — | — | — | — |
| Polyvinylpyrrolidone K25 | — | — | — | — | — | — | — | 1 |
| Polyvinylpyrrolidone K90 | 0.05 | — | — | — | — | — | — | — |
| Polyethylene glycol 6000 | — | — | — | — | — | — | 0.5 | — |
| Polyethylene glycol 4000 | 0.5 | — | — | — | — | — | — | — |
| Polyethylene glycol 400 | — | 1 | — | — | — | — | — | — |
| Hydroxyethyl cellulose | — | — | — | — | — | 0.2 | — | — |
| Hydroxypropylmethylcellulose | — | — | — | 0.01 | — | — | — | 0.2 |
| Carmellose sodium | — | 0.01 | — | — | — | — | — | — |
| I-menthol | — | — | — | 0.002 | — | 0.001 | 0.04 | — |
| d-camphor | — | — | — | 0.008 | — | — | — | — |
| dl-camphor | — | — | — | — | — | 0.015 | 0.02 | — |
| d-borneol | — | — | — | — | — | 0.01 | — | 0.02 |
| Geraniol | — | — | — | 0.005 | — | 0.005 | — | — |
| Eucalyptus oil | — | — | — | — | — | 0.005 | — | — |
| Bergamot oil | — | — | — | 0.002 | — | — | — | — |
| Cool mint oil | — | — | — | — | 0.01 | — | — | — |
| Mentha oil | — | — | — | — | — | 0.016 | — | — |
| Sodium hyaluronate | — | — | 0.3 | 0.001 | — | — | — | — |
| Alginic acid | — | — | — | — | 0.1 | — | — | — |
| Boric acid | — | — | 0.15 | — | 2 | 0.5 | 0.7 | 1.6 |
| Borax | — | — | 0.005 | — | 0.4 | 0.01 | 0.03 | 0.28 |
| Sodium dihydrogen phosphate-dihydrate | 0.5 | — | 0.05 | — | — | — | — | — |
| Disodium | — | 0.2 | 0.5 | — | — | — | — | — |
| hydrogenphosphate-dodecahydrate | | | | | | | | |
| Gellant gum | — | — | — | — | — | — | — | 0.02 |
| Propylene glycol | 2 | 1 | 0.3 | — | — | — | 0.8 | — |
| Sodium chloride | — | — | 0.2 | 0.5 | 0.2 | 0.1 | — | — |
| Potassium chloride | — | — | 0.01 | — | 0.02 | — | — | — |
| Sodium hydrogen carbonate | 0.2 | — | — | — | — | — | — | — |
| Magnesium sulfate | 0.001 | — | — | — | — | — | — | — |
| 10% alkylpolyaminoethylglycine liquid | — | — | — | — | — | — | — | 0.1 |
| Benzalkonium chloride | 0.01 | 0.01 | 0.003 | 0.01 | — | 0.01 | 0.0001 | 0.001 |
| Chlorhexidine glyconate | — | — | 0.002 | — | — | — | — | — |
| Potassium sorbate | — | — | — | — | — | — | — | 0.01 |
| Chlorobutanol | — | — | — | 0.2 | — | — | 0.3 | — |
| Edetate sodium | — | — | 0.005 | 0.08 | 0.05 | 0.008 | 0.08 | 0.06 |
| Dibutylhydroxytoluene | — | — | — | — | — | — | — | 0.005 |
| Phenethyl alcohol | — | — | — | 0.05 | — | — | — | — |
| Ethanol | — | — | 0.5 | — | — | — | — | — |
| Polyoxyethylene (20) sorbitan oleate | 0.5 | 0.5 | — | 0.2 | — | 0.2 | 0.5 | 0.3 |
| Polyoxyethylene hydrogenated castor oil 60 | 0.5 | 0.5 | — | — | 0.5 | — | 0.8 | 0.4 |
| Polyoxyethylene castor oil 10 | — | — | — | — | — | — | 1 | — |
| Polyoxyethylene castor oil 35 | — | — | 1 | — | — | — | — | — |
| Polyoxyethylene (200) polyoxypropylene | — | — | — | — | — | — | — | 0.1 |
| (70) block copolymer | | | | | | | | |
| Monostearin acid polyethylene glycol 4 | 0.8 | — | — | — | — | — | — | — |
| Monostearin acid polyethylene glycol 140 | — | 0.05 | 2 | — | — | — | — | — |
| Sesame oil | — | — | — | — | — | 0.08 | — | — |
| Castor oil | 0.001 | — | — | — | — | — | — | — |
| Hydrochloric acid | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Sodium hydroxide | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Purified water | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| pH | 8.8 | 4.5 | 7 | 6 | 8.8 | 5.5 | 5.2 | 7 |

TABLE 22

| | Prepa- | Prepa- | Prepa- | Prepa- | Prepa- | Prepa- | Prepa- | Prepa- |
|---|---|---|---|---|---|---|---|---|
| | ration | ration | ration | ration | ration | ration | ration | ration |
| | Example | Example | Example | Example | Example | Example | Example | Example |
| | 9 | 10 | 11 | 12 | 13 | 14 | 15 | 16 |
| White vaseline | 0.005 | 0.05 | 0.01 | 0.1 | 0.003 | 0.01 | 0.005 | 0.01 |
| Tetrahydrozoline hydrochloride | — | — | 0.01 | — | — | — | — | — |
| Neostigmine methylsulfate | — | — | 0.005 | — | — | — | — | — |
| Disodium cromoglycate | 2 | — | — | — | — | — | — | — |
| Chlorpheniramine maleate | 0.03 | — | 0.03 | — | 0.003 | — | — | — |
| Pranoprofen | 0.05 | — | — | — | — | — | — | — |
| Epsilon aminocaproic acid | 0.5 | — | 3 | — | — | — | — | — |
| Glicyrrhizic acid 2K | — | — | — | — | 0.025 | 0.25 | — | — |
| Berberine sulfate | — | — | — | — | 0.01 | — | — | — |
| Cyanocobalamin | — | — | — | — | 0.006 | — | — | — |
| Pyridoxine hydrochloride | — | — | 0.03 | — | 0.01 | — | — | — |
| d-α-tocopheryl acetate | — | — | 0.05 | — | 0.005 | — | — | — |
| Retinol palmitate | — | — | 35000 unit | — | — | — | — | — |
| Potassium aspartate | 0.1 | — | 0.2 | — | 0.1 | — | — | — |
| Aminoethylsulfonic acid | — | 0.5 | 0.1 | 0.1 | 0.1 | 1 | — | — |
| Sodium chondroitin sulfate | 0.5 | 0.5 | 0.05 | — | 0.01 | — | — | — |
| Polyvinylpyrrolidone K25 | — | — | — | 1.8 | — | — | 2.5 | — |
| Polyethylene glycol 400 | — | — | — | 1 | — | — | — | — |
| Hydroxyethyl cellulose | 0.01 | — | — | — | — | — | — | — |
| Hydroxypropylmethylcellulose | — | 0.02 | — | — | 0.2 | — | — | — |
| Carboxyvinyl polymer | 0.01 | — | — | — | — | — | — | — |
| Carmellose sodium | — | — | — | 0.4 | — | — | — | — |
| I-menthol | — | 0.01 | 0.005 | — | 0.004 | 0.01 | — | — |
| d-camphor | — | — | — | — | 0.004 | — | — | — |
| dI-camphor | — | — | 0.005 | — | 0.008 | 0.005 | — | — |
| d-borneol | — | — | 0.005 | — | 0.005 | 0.005 | — | — |
| Eucalyptus oil | — | — | 0.005 | — | — | — | — | — |
| Bergamot oil | 0.002 | — | — | — | 0.001 | — | — | — |
| Sodium hyaluronate | — | 0.005 | — | — | 0.005 | — | — | — |
| Arginine acid | — | — | — | 1 | — | — | — | — |
| Boric acid | 0.8 | 0.1 | 1.4 | 0.4 | 1.6 | 1 | 0.9 | — |
| Borax | — | 0.1 | — | 0.03 | 0.04 | 0.03 | 0.5 | — |
| Sodium dihydrogen phosphate-dihydrate | — | — | — | — | — | — | — | 0.1 |
| Disodium | — | 0.3 | — | — | — | — | — | 1.5 |
| hydrogenphosphate-dodecahydrate | | | | | | | | |
| Sodium citrate hydrate | — | 0.5 | — | — | — | — | — | — |
| Anhydrous citric acid | — | 0.01 | — | — | — | — | — | — |
| Trometamol | 0.01 | — | 0.1 | — | — | 1 | — | 0.5 |
| Glucose | — | 0.001 | — | — | — | — | — | — |
| Propylene glycol | 1.5 | — | 0.3 | 2.4 | 0.1 | 0.5 | — | — |
| Glycerin | — | — | — | — | 0.1 | 2.1 | 0.8 | 0.1 |
| D-sorbitol | — | — | — | 0.5 | — | — | — | — |
| Sodium chloride | — | 0.4 | 0.2 | — | — | — | — | — |
| Potassium chloride | — | 0.1 | — | — | — | — | — | — |
| Calcium chloride | — | 0.016 | — | — | — | — | — | — |
| Magnesium sulfate | — | 0.01 | — | — | — | — | — | — |
| 10% alkylpolyaminoethylglycine liquid | — | — | 0.05 | — | — | — | — | — |
| Hydrochloric-acid polyhexamethylene | — | 0.00002 | — | 0.00008 | — | — | — | — |
| biguanide | | | | | | | | |
| Benzalkonium chloride | — | — | 0.01 | — | — | 0.01 | 0.005 | 0.01 |
| Potassium sorbate | — | 0.01 | — | — | — | — | — | — |
| Chlorobutanol | — | — | 0.3 | — | — | 0.2 | — | — |
| Zinc chloride | — | — | — | 0.0004 | — | — | — | — |
| Edetate sodium | 0.08 | 0.13 | 0.1 | — | 0.01 | 0.1 | 0.05 | — |
| Dibutylhydroxytoluene | 0.005 | — | 0.005 | — | — | — | — | — |
| Polyoxyethylene (20) sorbitan oleate | 0.6 | 0.2 | 0.05 | 0.3 | 0.01 | 1 | 0.3 | 1 |
| Polyoxyethylene hydrogenated castor oil 60 | — | 0.4 | 0.5 | — | 0.4 | — | — | — |
| Polyoxyethylene hydrogenated castor oil 40 | — | 0.2 | — | — | — | — | — | — |
| Polyoxyethylene castor oil 3 | — | — | — | 0.1 | — | — | — | — |
| Polyoxyethylene castor oil 35 | — | — | — | — | — | 0.6 | — | — |
| Polyoxyethylene (196) polyoxypropylene | — | 0.01 | — | — | — | — | — | — |
| (67) block copolymer | | | | | | | | |
| Polyoxyethylene (200) polyoxypropylene | — | — | 3 | — | — | — | — | — |
| (70) block copolymer | | | | | | | | |
| Monostearin acid polyethylene glycol 40 | — | — | — | 1 | — | — | — | — |
| Ketotifen fumarate | — | — | — | — | — | 0.069 | — | — |
| Tranilast | — | — | — | — | — | — | 0.5 | — |
| Pemirolast potassium | — | — | — | — | — | — | — | 0.1 |
| Cyclodextrin | — | — | 0.1 | — | — | — | — | — |
| D-mannitol | — | — | — | — | — | 1 | — | — |
| Hydrochloric acid | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Sodium hydroxide | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Purified water | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| pH | 7.2 | 7.5 | 6.5 | 6.5 | 6 | 5.3 | 7.5 | 7.8 |

TABLE 23

| | Prepa- | Prepa- | Prepa- | Prepa- | Prepa- | Prepa- | Prepa- | Prepa- |
|---|---|---|---|---|---|---|---|---|
| | ration | ration | ration | ration | ration | ration | ration | ration |
| | Example | Example | Example | Example | Example | Example | Example | Example |
| | 17 | 18 | 19 | 20 | 21 | 22 | 23 | 24 |
| White vaseline | 0.05 | 0.01 | 0.05 | 0.1 | 0.01 | 0.005 | 0.1 | 0.05 |
| Epsilon aminocaproic acid | — | — | 3 | — | — | — | — | — |
| Polyvinylpyrrolidone K25 | 1.2 | — | — | — | — | — | — | — |
| Polyvinylpyrrolidone K30 | — | — | — | — | — | — | 0.5 | — |
| Hydroxyethyl cellulose | — | 0.8 | — | 0.4 | — | — | — | — |
| Carboxyvinyl polymer | — | — | — | — | — | — | — | 0.1 |
| Boric acid | 1.7 | — | — | 0.4 | — | 1 | 1.4 | — |
| Borax | 0.2 | — | — | — | — | — | 0.3 | — |
| Sodium dihydrogen phosphate-dihydrate | — | — | — | 0.01 | 0.2 | 0.06 | — | — |
| Disodium | — | — | — | 0.1 | 0.8 | 0.7 | — | — |
| hydrogenphosphate-dodecahydrate | | | | | | | | |
| Sodium citrate hydrate | — | — | — | 1 | — | — | — | — |
| Anhydrous citric acid | — | — | — | 0.1 | — | — | — | — |
| Trometamol | — | — | — | 2.5 | — | — | — | — |
| Propylene glycol | — | — | 0.3 | 1 | — | — | — | — |
| Glycerin | — | — | — | 1.5 | — | — | — | — |
| Sodium chloride | — | 0.9 | — | 0.2 | 0.5 | 0.1 | — | 0.2 |
| Benzalkonium chloride | — | 0.01 | — | 0.005 | 0.01 | — | 0.01 | 0.01 |
| Chlorobutanol | — | — | 0.4 | — | — | — | — | — |
| Edetate sodium | — | — | — | 0.1 | — | 0.05 | 0.01 | 0.1 |
| Monoethanolamine | — | — | 1 | — | — | — | — | — |
| Polyoxyethylene (20) sorbitan oleate | 0.5 | 1 | 0.5 | 0.5 | 1 | 1 | 1 | 1 |
| Polyoxyethylene hydrogenated castor oil 60 | 1 | — | 0.5 | 1 | — | — | — | 1 |
| Polyoxyethylene castor oil 10 | 0.1 | — | — | — | — | — | — | — |
| Polyoxyethylene castor oil 35 | — | — | 0.01 | 0.01 | — | — | — | — |
| Amlexanox | 0.25 | — | — | — | — | — | — | — |
| Ibudilast | — | 0.01 | — | — | — | — | — | — |
| Acitazanolast | — | — | 0.1 | — | — | — | — | — |
| Levocabastine hydrochloride | — | — | — | 0.025 | — | — | — | — |
| Olopatadine hydrochloride | — | — | — | — | 0.7 | — | — | — |
| Epinastine hydrochloride | — | — | — | — | — | 0.05 | — | — |
| Bromfenac sodium | — | — | — | — | — | — | 0.1 | — |
| Nepafenac | — | — | — | — | — | — | — | 0.1 |
| D-mannitol | — | — | — | 2 | — | — | — | 4 |
| Tyloxapol | — | — | — | — | — | — | — | 0.05 |
| Hydrochloric acid | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Sodium hydroxide | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| Purified water | Appro- | Appro- | Appro- | Appro- | Appro- | Appro- | Appro- | Appro- |
| | priate | priate | priate | priate | priate | priate | priate | priate |
| | amount | amount | amount | amount | amount | amount | amount | amount |
| pH | 7.3 | 6.2 | 5.3 | 7 | 7 | 7 | 8.3 | 7.4 |

1. An aqueous ophthalmic composition comprising:
(A) vaseline; and
(B) at least one agent selected from the group consisting of a vinyl-based polymer compound, a saccharide, an amino acid, polyalcohol, a preservative, a sulfa drug, a vitamin, an inorganic salt compound, an ocular muscle modulator component, a vasoconstrictor, a stabilizer, polyoxyethylene polyoxypropylene glycol, and a vegetable oil.

2. The aqueous ophthalmic composition according to claim 1, wherein the saccharide is selected from the group consisting of methyl cellulose, hydroxyethyl cellulose, hydroxypropylcellulose, hydroxypropylmethylcellulose, carboxymethyl cellulose, hyaluronic acid, chondroitin sulfate, alginic acid, dextran, gellant gum, salts thereof and glucose;
the vinyl-based polymer compound is selected from the group consisting of polyvinyl alcohol, polyvinylpyrrolidone, and a carboxyvinyl polymer;
the polyoxyethylene polyoxypropylene glycol is selected from the group consisting of Poloxamer 188 and Poloxamer 407;
the polyalcohol is selected from the group consisting of glycerin, propylene glycol, polyethylene glycol, and mannitol;
the inorganic salt is selected from the group consisting of sodium chloride, potassium chloride, calcium chloride, zinc chloride, and magnesium sulfate;
the vegetable oil is selected from the group consisting of sesame oil and castor oil;
the amino acid is selected from the group consisting of aspartic acid, aminoethylsulfonic acid, or salts thereof;
the vitamin is selected from the group consisting of flavin adenin dinucleotide sodium, panthenol, sodium pantothenate, calcium pantothenate, pyridoxine hydrochloride, cyanocobalamin, retinol acetate, retinol palmitate, and tocopherol acetate;
the ocular muscle modulator component is neostigmine or a salt thereof;
the vasoconstrictor is selected from the group consisting of tetryzoline, naphazoline, and salts thereof;
the preservative is selected from the group consisting of alkyldiaminoethylglycine hydrochloride, benzalkonium chloride, benzethonium chloride, chlorhexidine glyconate, chlorobutanol, sorbic acid, potassium sorbate, sodium dehydroacetate, methyl parahydroxybenzoate, ethyl parahydroxybenzoate, propyl parahydroxybenzoate, butyl parahydroxybenzoate, and a biguanide compound; and
the stabilizer is selected from the group consisting of dibutylhydroxytoluene, trometamol, and monoethanolamine; and
the sulfa drug is sulfisomezole or sulfamethoxazole sodium.

3. The aqueous ophthalmic composition according to claim 1, further comprising (C) a nonionic surfactant.

4. The aqueous ophthalmic composition according to claim 1, further comprising (D) a buffer.

5. The aqueous ophthalmic composition according to claim 1, wherein the content of (A) the vaseline is from 0.00001 to 10 w/v % based on the total amount of the aqueous ophthalmic composition.

6. The aqueous ophthalmic composition according to claim 1, wherein the total content of the component (B) is from 0.00001 to 50000 parts by weight based on 1 part by weight of the total content of the component (A).

7. The aqueous ophthalmic composition according to claim 1, comprising 50% by weight or more of water relative to the total amount of the aqueous ophthalmic composition.

8. The aqueous ophthalmic composition according to claim 1, wherein the composition is contained in a plastic container.

9. The aqueous ophthalmic composition according to claim 1, wherein the composition is formulated for use with used for contact lenses.

10. The aqueous ophthalmic composition according to claim 1, wherein the composition is formulated for reducing ocular discomfort.

11. The aqueous ophthalmic composition according to claim 1, wherein (A) the vaseline is white vaseline.

12. The aqueous ophthalmic composition according to claim 1, wherein the composition is a liquid drug or a gel drug.

13. The aqueous ophthalmic composition according to claim 1, wherein the total content of the component (B) is from 0.0000001 to 25 w/v % based on the total amount of the aqueous ophthalmic composition.

14. The aqueous ophthalmic composition according to claim 4, wherein the total amount of the component (D) is from 0.0001 to 30000 parts by weight based on 1 part by weight of the total amount of the component (A).

15. The aqueous ophthalmic composition according to claim 1, further comprising (E) a terpenoid compound.

16. The aqueous ophthalmic composition according to claim 15, wherein the total content of the component (E) is from 0.00001 to 10 w/v % based on the total amount of the aqueous ophthalmic composition.
